(19) United States
(12) Patent Application Publication
(10) Pub. No.: US 2025/0244318 A1
(43) Pub. Date: Jul. 31, 2025

(54) ANALYTE DETECTION SYSTEMS AND METHODS OF USE

(71) Applicant: Massachusetts Institute of Technology, Cambridge, MA (US)

(72) Inventors: Emily Barret Hanhauser, Brookline, MA (US); Mary Claire Strawser, Cambridge, MA (US); Chun Man Chow, Cambridge, MA (US); Krithika Ramchander, Cambridge, MA (US); Rohit N. Karnik, Cambridge, MA (US)

(73) Assignee: Massachusetts Institute of Technology, Cambridge, MA (US)

(21) Appl. No.: 18/703,414

(22) PCT Filed: Oct. 24, 2022
(86) PCT No.: PCT/US2022/047622

§ 371 Date: Apr. 22, 2024

Related U.S. Application Data
(60) Provisional application No. 63/271,596, filed on Oct. 25, 2021.

(51) Int. Cl. G01N 33/543; G01N 15/06; G01N 15/1433

(52) U.S. Cl. CPC G01N 33/54313; G01N 15/0612; G01N 15/1433

(57) ABSTRACT

Analyte detection systems and related methods are generally described. In some embodiments, an analyte testing system may rapidly and sensitively sense analyte (e.g., any biological and/or chemical analyte) from a testing sample in a fluid container, which may also include one or more populations of particles coated with/including binding ligands, moieties, and/or coatings for binding to analyte, if present. An external force may be applied to the container to urge the particles to move through the fluid faster than a rate determined by diffusion. The particles may subsequently settle on a target surface. The particles disposed on the surface may be characterized to determine the presence of the analyte. In some embodiments, a sequence of images of particles bound specifically and nonspecifically to the surface may be analyzed to determine a relative displacement of the particles, and subsequently, analyte concentration of the testing sample.

### CROSS-REFERENCE TO RELATED APPLICATIONS

This application claims the benefit under 35 U.S.C. § 119 (e) of U.S. provisional application Ser. No. 63/271,596, filed Oct. 25, 2021, the disclosure of which is incorporated by reference in its entirety.

### FIELD

This technology is generally related to analyte detection systems and related methods. More specifically, systems and methods to maximize detection sensitivity in a simple to operate and low-cost manner are described.

### BACKGROUND

To protect human, animal, and environmental health, diverse applications rely on the ability to accurately detect and quantify biological and/or chemical analytes. Results of these tests may inform decisions on the individual, group, and societal level. For example, 14 billion diagnostic or laboratory tests are performed in the U.S. each year to inform treatment of human diseases, including cancers, autoimmune and metabolic disorders, genetic conditions, and infectious diseases. In the context of pandemic diseases, such as COVID-19, detection assays were used for patient diagnosis and monitoring, public health screening and research for disease understanding and therapeutic or vaccine development.

Some cases, such as with food poisoning outbreaks, which cost an estimated $15 billion per year and are generally due to pathogenic bacteria, it may be desirable to use assays to detect the causative pathogen as well as trace the spread and determine the source. Similar testing needs arise in detecting biological water contamination, a major cause of diarrheal diseases, a leading cause of death for children under five years old worldwide, as well as in the treatment and understanding of animal diseases and zoonoses.

### SUMMARY

In some embodiments, a method of analyte detection includes: providing a volume having a first plurality of particles; introducing a first analyte solution to the volume, wherein the first plurality of particles are configured to react with a first analyte when present in the first analyte solution; applying an external force to the volume to urge the first plurality of particles to move through the first analyte solution; and allowing the first plurality of particles to settle on a first portion of a target surface, wherein the first portion of the target surface is configured to bind the first plurality of particles when the first analyte is present in the first analyte solution.

In some embodiments, a method of analyte detection includes: obtaining a sequence of images of a surface in a volume, the surface having a first plurality of particles disposed on the surface, wherein the first plurality of particles are configured to react with a first analyte when present, and wherein the surface is configured to bind to the first plurality of particles when the first analyte is present; measuring relative displacement of the first plurality of particles based at least in part on the sequence of images; and determining the presence of the first analyte based at least in part on the relative displacement of the first plurality of particles.

In some embodiments, a system for analyte detection includes: a volume configured to contain a first analyte solution; a first plurality of particles arranged in the volume, the first plurality of particles configured to react with a first analyte when present in the first analyte solution; and a first target surface configured to bind with the first plurality of particles when the first analyte is present in the first analyte solution, and wherein the first plurality of particles is configured to move through the first analyte solution toward the first target surface.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1B depict an analyte testing system, according to some embodiments;

FIGS. 2A-2D depict various embodiments of a target surface for the analyte testing system depicted in FIGS. 1A-1B;

FIGS. 3A-3D depict a method of operating an analyte testing system, according to some embodiments;

FIGS. 4A-4B depict a method of detecting and analyzing analyte in a testing system, according to some embodiments;

FIGS. 5A-5E show exemplary particle motion data vs. analyte concentration, according to some embodiments

FIG. 6A is a flow chart of a method of operating an analyte testing system, according to some embodiments;

FIG. 6B is a flow chart of a method of detecting and analyzing analyte in a testing system, according to some embodiments;

FIG. 7 shows a plot of particle displacement on a target surface of an analyte testing system vs. analyte concentration, according to some embodiments;

FIG. 8 shows a plot of number of particles on a target surface vs. magnitude of external force applied, according to some embodiments;

FIG. 9 shows a plot of analyte assay time vs. particle diameter and concentration, according to some embodiments.

### DETAILED DESCRIPTION

Biological and chemical sensing targets may be classified into relatively few groups, which range in size, make-up, complexity, and stability. Often, several analyte targets may be indicative of analyte presence, i.e., virus detection by either sensing the whole virus particle or by sensing viral nucleic acid. Gold-standard methods for detection and enumeration generally require a sample preparation or concentration step to isolate the target from the background matrix, followed by a sensing method, the form of which depends on the specific analyte of interest, as summarized in table 1 below.

TABLE 1
summary of conventional detection
methods for exemplary analyte types

| Analyte Type | Conventional Detection Method |
|---|---|
| Nucleic acid | Polymerase chain reaction and versions thereof |
| Protein or | Enzyme-linked immunosorbent assay, |
| molecular | mass spectrometry |
| Whole pathogen | Culture on solid or liquid media |
| Whole cell | Flow cytometry |

While the conventional methods outlined in Table 1 above may possess the necessary sensitivity, specificity and cost for many applications, they are typically restricted to specialized laboratories with high operational costs, with their requirements of complex instrumentation, cold chain reagents, and personnel experience. As such, the Inventors have recognized that these conventional techniques have limited applicability in point-of-care applications, infrastructure-poor areas such as rural or resource-constrained settings, and for mass screening or monitoring campaigns.

In some instances, systems such as lateral flow assays (LFAs) and other sandwich assays have increased the reach of testing, offering rapid results in a cheap, simple, room-temperature stable and easy-to-operate format. However, LFAs are not usually quantitative and have lower sensitivities than their gold-standard counterparts. As a result, the Inventors have recognized that significant gaps in widely applicable, high sensitivity testing methods, for molecular and especially for particulate analytes, remain. In particular, the trade-off between sensitivity, speed, and cost have limited the scope and accessibility of biological and/or chemical testing. For example, since the late 2019 emergence of COVID-19, over 600 million cases have been recorded worldwide, causing the need for diagnostic testing to vastly outstrip the supply of the conventional gold-standard test, reverse transcription polymerase chain reaction (RT-PCR), which can detect low numbers of viral RNA within several hours. Despite the test itself having relatively quick time-to-result, delays in PCR testing have arisen due to sample transport to an appropriate lab, reagent shortages, and high demand. Despite their sensitivity, RT-PCR costs range from $20-850 depending on the healthcare facility and insurance structure. Lower cost ($5-50/test) rapid (<30 minutes) antigen tests have been used to complement molecular testing; however, they are less sensitive than RT-PCR tests, particularly during the patient's symptomatic phase.

In view of the above, the Inventors have recognized the benefits associated with low-cost, high sensitivity point-of-care testing systems for chemical and/or biological sensing. In some cases, the testing system may minimize fluid handling of testing samples such that the testing systems may be used without the need for complex and cost-prohibitive laboratory equipment. However, instances in which different benefits are offered by the systems and methods disclosed herein are also possible.

In some embodiments, a testing system may rapidly and sensitively sense analyte (e.g., any biological and/or chemical analyte, as described below) in a low-cost manner. The testing systems of the present disclosure may employ one or more populations of particles which may be loosely arranged in a fluid container. A fluid testing sample (e.g., saliva, or others, as described below) potentially having one or more populations of biological and/or chemical analytes may be introduced into the system, allowing the analyte(s) to react with the particles, which may be coated with one or more affinity agents. In some embodiments, the particles may be able to move through the sample fluid in order to bind and/or react with as many analytes as possible. The particles may be urged through the fluid by an externally applied force, such as by gravity or other appropriate type of force as detailed further below. For example, in some embodiments, the testing sample may be introduced into the container, the container may be sealed, and subsequently agitated to urge the particles to move through the fluid. In some instances, the particles may be of an appropriate size and density such that gravity may cause the particles to settle out of the solution. In this way, the particles may be exposed to a larger volume of fluid including the potential analyte(s) as compared to particles that move using diffusion-based mechanisms where the particles undergo less overall motion.

In some embodiments, the concentration of particles following mixing with the sample may be less than or equal to approximately 106, 105, 104, 103, 100, and/or any other number of particles per mL. The concentration of particles following mixing with the sample may also be greater than or equal to 100, 103, 104, 105, 106, and/or any other number of particles per mL. Combinations of the foregoing, including particle concentrations between 100 and 106 per mL are also contemplated, as well as particle concentrations above and below the ranges listed above, as the present disclosure is not limited by the particle concentration.

When exposed to the external force, the particles may settle onto a target surface within the container. In some embodiments, as will be described in greater detail below, the analyte bound to the particles may react with affinity agents on the target surface to attract and subsequently bind the particles to the target surface. The target surface populated with settled particles may then be visualized using a reader compatible with the container, in order to identify the particles and their relative motion.

In some embodiments, the particles may move through any suitable distance within the container to arrive at the target surface. The particles may traverse a distance of greater than or equal to approximately 0.1 mm, 1 mm, 10 mm, 1 cm, 10 cm, and/or any other distance before arriving at a target surface. The particles may also traverse a distance of less than or equal to approximately 10 cm, 1 cm, 10 mm, 1 mm, 0.1 mm, and/or any other distance before arriving at a target surface. It should be appreciated that the particles may traverse any suitable distance within the fluid of the container as the present disclosure is not limited by the path of the particles prior to arriving at the target surface.

In some embodiments, the reader may include a photosensitive detector such as a high-resolution camera, a combined microscope and camera, a mobile device, a smartphone, a waveguide integrated into the target surface to detect scattered or absorbed light, a magnetic field detector, surface acoustic wave sensor, microscope, electrochemical sensor, and/or any other appropriate type of sensor or combination of sensors capable of sensing the particles on a target surface. For example, in some embodiments, the reader may collect one or more images of the surface, and in some instances a sequence of images. In some instances where a sequence of images is collected, an image analysis system may use the sequence of images to determine dynamic properties of the particles bound to the surface. For example, one or more particles may be identified in the sequence of images, and their respective trajectories over time may be tracked. As will be described in greater detail below, the dynamic behavior of the particles through the sequence of images may be processed to determine whether or not analyte is present in the testing sample, analyte concentration bound to the particles, which may subsequently be used to determine concentration in the testing sample, and/or analyte type. Alternatively, the presence and/or number of particles on a surface without regard to movement of the particles on the surface may be used to determine the presence of an analyte as the disclosure is not limited to the specific analysis method. In some embodiments, sensors may be integrated into the target surface. For example, electrodes may be patterned on the target surface for electrochemical detection, or plasmonic features may be included on the target surface to facilitate detection or analysis by optical means. In some embodiments, fiducial markings may be included on the target surface to facilitate localization or optical focusing by the reader.

As with most biological and/or chemical sensing assays, the Inventors have recognized the challenges associated with nonspecific binding of particles/analyte on the target surface. Nonspecific binding may refer to particles/analyte statically or otherwise settled on a target surface without having a specific surface affinity agent-particle/analyte bond. In some embodiments, van der Waals forces or other surface forces may bond a particle/analyte to the target surface in absence of a binding site. As such, the Inventors have recognized that the presence of such nonspecifically bound particles/analytes may occlude data regarding the concentration of the testing sample.

In view of the above, the testing systems of the present disclosure may employ one or more detection techniques to help distinguish specific vs. nonspecific binding in order to quantify the concentration of analyte in the testing solution more accurately. In some embodiments, processes may be employed to dislodge or disengage at least a subset of the nonspecifically bound population of analyte from the target surface, as will be described in greater detail below.

In some embodiments, the dynamic behavior of the particles bound to the surface, which may be referred to as the particles' Brownian motion, or the random movement of the particle due to collision with surrounding molecules, may be examined in order to quantify the amount of analyte in the testing sample. It should be appreciated that the Brownian motion arises naturally without external actuation and may be monitored by the reader. The trajectory and extent of the particle motion relative to the stationary target surface may reflect its analyte-binding status and be used to determine the amount of analyte in a sample. In some embodiments, such measurements may also reflect non-specific binding between the particle and the target surface. For example, a particle which is not chemically tethered to the surface through a targeted affinity agent (e.g., non-specifically bound) may traverse a larger distance than a particle that is bound to the surface through an analyte. Similarly, a particle which is tethered to the surface by multiple bound analytes may traverse a smaller distance than a particle that is tethered to the surface via a single bound analyte. In this way, the Brownian motion of the particles on the surface may determine analyte presence and/or concentration in the testing sample.

In some embodiments, image analysis (e.g., digital data analysis, including image analysis algorithms, statistical methods, or machine learning) may be applied to the observations of particle motion in order to distinguish specifically vs nonspecifically bound particles on the target surface. The motion of the particles on the target surface may be quantified using any suitable metric, including, but not limited to, average velocity, mean squared distance (MSD), root mean squared distance (RMSD), the radius of a circle or radii of an ellipse which encompasses the entire particle trajectory, combinations thereof, and/or any other method of quantifying particle movement (e.g., displacement) from a sequence of images. It should be appreciated that the motion or combination of motions of the particles, including random Brownian-like motion, motion induced by fluid flow, gravity, actuation in response to one or more external force, and other means may be used to infer the presence of target analytes that influence the interaction of the particles with the target surface. In some embodiments, the number or motion of aggregates of two or more particles on the target surface or the patterns formed by spatial distribution of particles on the target surface may be used to quantify the analyte.

In some embodiments, the quantification method may be coupled with other actuation methods to enhance assay sensitivity. For example, the surface may be inverted to promote removal of untethered particles by gravity and the Brownian motion of the remaining surface-tethered particle may be monitored. Temporal information may be collected and used to infer the presence of the analyte and information regarding the binding between the analyte and the affinity agents. For example, over long enough observation periods, analyte may dissociate from the particle or the surface, resulting in a change in particle motion. Observation and analysis of this phenomenon may yield an estimate of the dissociation constant of the analyte-affinity agent complex.

It should be appreciated that in some embodiments, depending on the binding chemistries of the affinity agent (e.g., antibodies, antigens, aptamers, etc.) employed, some concentration of analyte may be bound to the target surface without being bound to a particle. In some embodiments, the affinity agent may be in the form of moieties, ligands, and/or coatings. In some embodiments, the geometry of the particles (e.g., spherical) relative to the target surface (e.g., flat) may enable analyte to bind to the surface in the interstitial spaces between particles. However, it should also be appreciated that the testing systems herein may, in some embodiments, employ readily available, low cost and/or low power readers (e.g., cameras used in mobile devices). Therefore, these cameras may not be sufficiently high resolution to identify individually bound analyte. As such, the particles, which may be larger than the analyte and more readily visible with low cost readers, may serve as a marker of the analyte concentration. In this way, the testing systems may benefit from high sensitivity while reducing the operating costs. In some embodiments, the particles may be selected to be easily distinguishable from any background particulates that may be present using methods including, but not limited to, color, fluorescence, luminescence, reflectivity, incorporation of plasmonic structures to scatter light, and/or other methods which may distinguish particles settled on the surface vs. particles in the background fluid.

As described previously, the particles may be urged to move through the fluid of the testing sample upon exposure to an external force. In this way, the particles may be exposed to a larger population of analyte within the fluid, and may arrive at the target surface for analysis faster than diffusion-limited particles. The external force may be any category of force, including forces applied to a body and/or surface of the particles. In some embodiments, the external force may be an external agitation force which serves to disperse, mix, and subsequently move the particles through the testing sample fluid. The agitation force may be a user shaking or agitating the container or any form of mechanical and/or automated stirring, shaking, rotating, shearing, vortexing, vibrating, centrifuging, and/or mixing mechanism. In some embodiments, the external force may be gravitational, electrokinetic, electrohydrodynamic, dielectrophoretic, acoustic, magnetohydrodynamic, thermal convection, optical, radiation, magnetic, bubbling of gas injected or produced by a reaction, surface-tension-driven flow, combinations thereof, and/or any other external force instead of or in combination with mechanical mixing.

In some embodiments, a force (e.g., an applied magnetic field or acoustic field) may be used to disperse and mix the particles through the testing sample fluid, and subsequently turned off to allow another external force (e.g., gravity) to urge the particles toward the target surface. Accordingly, more than one force may be employed to direct particles through the testing sample fluid. It should be appreciated that the same type of force may be employed in two directions to direct particles through the fluid. For example, the container may be turned upside down following introduction of testing sample into the container to drive the particles through the fluid with gravity, and turned back right side up in order to drive the particles toward the target surface. Accordingly, an external force (e.g., gravity) may be used in more than one way.

In some embodiments, a secondary external force may be employed to disengage non-specifically bound particles from the target surface. For example, paramagnetic particles may be driven through the testing sample fluid through an external magnetic field applied to the container. Following sufficient mixing of the particles within the fluid, the force may be adjusted to allow the particles to settle on the target surface. The container may then be inverted to subject the particles to gravity, allowing a first population of non-specifically bound particles to disengage from the surface. The container may subsequently be exposed to vibration to further disengage non-specifically bound particles. It should be appreciated that the secondary force(s) may be controlled such that they are not sufficiently strong to disengage the chemical bonds between the analyte/particle and the target surface, and the remaining population of particles on the target surface are mostly specifically bound particles.

In some embodiments, the secondary external force (e.g., magnetic actuation) may be used to interact with particles during or after particle-surface interaction. For example, under the influence of a magnetic field, weakly- or untethered particles may be lifted from the surface, twisted or rotated, dragged across the surface, or otherwise moved relative to the surface in various ways which may aid in differentiation of specific vs. nonspecifically bound particles. Depending on the embodiment, the magnet may be placed above or below the surface in order to levitate paramagnetic or diamagnetic particles, respectively. Magnetic actuation may be applied temporarily or remain in place for the entirety of the assay. Additionally, magnetic actuation may be applied to the whole surface or to a specific area of the surface.

It should be appreciated that any type or combination of types of external forces may be employed to urge the particles through the fluid, direct the particles to the target surface, and disengage non-specifically bound particles from the target surface, as the present disclosure is not limited by the external force(s) employed.

The testing systems of the present disclosure may be highly sensitive without employing complex and costly equipment. It should be appreciated that the sensitivity of the systems described herein may be determined by a variety of factors, including, but not limited to, the binding energy of the analyte with the affinity agent on the target surface, the binding energy between the analyte and the particle(s), the concentration of analyte within the testing sample, the method of visualizing the target surface (e.g., fluorescence, brightfield), any operating parameter of the reader (and its constituent components such as the camera, microscope, processors, etc.), fluid dynamic properties of the testing sample (e.g., viscosity, density, etc.), various properties of the particles (e.g., material composition, density, porosity, etc.), various properties of the analyte, and/or any other factor.

In some embodiments, the sensitivity of the testing systems and methods described herein may be greater than or equal to 1 pg/mL, 5 pg/mL, 10 pg/mL, 100 pg/mL, 250 pg/mL, 500 pg/mL, 1 ng/mL, and/or any other suitable sensitivity. The sensitivity of the testing systems may also be less than or equal to 1 ng/mL, 500 pg/mL, 250 pg/mL, 100 pg/mL, 10 pg/mL, 5 pg/mL, 1 pg/mL, and/or any other suitable sensitivity. Combinations of the foregoing, including testing systems having sensitivities between 1 pg/mL and 100 pg/mL are also contemplated. In some embodiments, the testing systems described herein may have molecular-level sensitivity. In some embodiments, the testing systems described herein may have sensitivities above, and/or below those outlined above. As such, the present disclosure is not limited by the sensitivity of the testing systems.

In some embodiments, the testing systems of the present disclosure may be employed to detect more than one type/class of analyte in a given testing sample in a multiplexing application. This may enhance the efficiency of the system, yielding data from more than one analyte in the same testing system. In some embodiments, a testing sample fluid having more than one target analyte type may be introduced into the testing container, which may include more than target surface and/or more than one particle population. Each particle population and target surface may be associated with a different analyte type, such that a first population of particles may bind to the first population of analyte and subsequently settle on a first target surface, and the second population of particles may bind to the second population of analyte from the test solution and subsequently settle on a second target surface (and so on, for testing samples with more than two analytes). In some embodiments, the entirety or a portion of the volume of the container may be divided into smaller sub-containers, with each sub-container having a target surface with a different functionalization. In some embodiments, each sub-container may be pre-loaded with particles with different functionalizations in the different sub-containers. The sub-containers may have any suitable size corresponding with the main container size, including dimensions between 10 μm to 10 cm, although dimensions above and below this range are also contemplated. In some embodiments, the sub-containers may have dimensions of 1 mm×1 mm×1 cm. A container may have any suitable number of sub-containers, including, but not limited to, greater than or equal to 1, 2, 5, 10, 20, 100, and/or any other suitable number of sub-containers. In some embodiments, the sidewalls of the sub-containers may have pores which allow fluids (e.g., air and water) to pass through, but limit the passage of particles between containers.

In some embodiments, a single population of particles may be employed to detect more than one analyte type. The particles may have more than one affinity agent so that it may bind to more than one analyte type. In some embodiments, a single target surface may be employed in multiplexing applications, having one or more areas and/or affinity agent for binding to the one or more analytes.

In multiplexing embodiments where more than one particle type is used, the particles may differ in size, shape, pattern, color, material composition, optical properties, and/or some other functionality that enables particles bound to the surface(s) to be distinguishable from each other. In some exemplary embodiments, a magnetic particle functionalized with one ligand may be distinguished from another magnetic or non-magnetic particle with a different magnetic response functionalized with another ligand by observing the motion of particles during the application of a changing magnetic field when the particles are moving toward or are on the target surface. In some embodiments, multiplexing may be achieved by patterning the target surface with different affinity agents enabling particles bound to different populations of analyte to be detected separately. For example, the surface may have two regions, the first region coated with an affinity molecule that binds only to analyte A, and the second region coated with an affinity molecule that binds only to analyte B. Thus, analyte A may be sensed by detecting the interaction of particles with the first region, and analyte B may be sensed by detecting the interaction of particles with the second region. The regions may be located in parallel such that some particles arrive at one region and other particles arrive at another region; the regions may also be placed in series such that particles first interact with one region and then with another region (if they are not arrested in the first region).

In some embodiments, multiplexing may be achieved by use of particles that experience different rates of motion through the fluid or across the surface. For example, in the case of gravity-driven motion of particles, denser particles may be coated with affinity molecules that bind to analyte A, whereas less dense particles may be coated with affinity molecules that bind to analyte B. The density difference may cause the denser particles to arrive earlier (on average) to the surface, and therefore, monitoring of the particle-surface interactions over time may be used to distinguish between the two analytes.

In embodiments where particles of different material compositions or structures are employed, more than one external force may be employed to drive each population of particles through the fluid and subsequently, toward the target surface.

In multiplexing applications, the testing systems may employ more than one population of particles to facilitate detection of more than one analyte. In some embodiments, the particles may differ in shape, size, optical property, and/or any other property in order to be visually distinguishable with the reader. For example, two populations of particles, each having a different fluorescence peak, may be employed to detect two populations of analyte in the testing sample.

In some exemplary embodiments, the target analytes may be cells distinguished by the presence of two or more antigens. To detect a cell that expresses both antigen A and antigen B, the particles may be coated with affinity agents (e.g., antibodies or affinity molecules) that bind to antigen A, and the target surface may be coated with antibodies or affinity molecules that bind to antigen B. Therefore, binding of particles to the target surface may occur only when cells expressing both antigens are present. Another approach may be to use particles that bind to either or both antigens A and B (or even a different antigen), and to arrange two surfaces in series where one is coated with an affinity molecule that binds to A and the other with an affinity molecule that binds to B. In this way, particles that may not be bound to the first target surface may interact with the other. In the same device, another pair of surfaces may be included with the reverse order (i.e., B upstream and A downstream), and additional surfaces may be included with affinity molecules that bind to only A or only B. Comparing binding of particles on such surfaces may provide a means to detect cells that express both antigens A and B. For example, if a surface that binds to antigen A is arranged in series with a surface that binds to antigen B, cells that express both antigens A and B will be arrested on the first surface and there may be few or no particles bound to the second surface. However, if the sample includes cells that express either antigen A or B, then there will be binding on both surfaces. A third approach may be to include a label (e.g., fluorescence, colored particle, enzyme, etc.) that binds to a specific antigen that can be detected by a reader. A fourth approach is to use weak adhesions, such as in cell rolling, and to alter the interaction or trajectories of individual particles as they interact with surfaces in series that are coated with affinity molecules to different antigens. For example, particles may be coated with affinity molecule binding to antigen A, and interact with two surfaces coated with affinity molecules binding antigens B and C, respectively. Patterns on the surface (chemical and/or geometric), combined with weak adhesive interactions, may be used to direct particles along different trajectories and to thereby affect their separation. The trajectories of the particles may be detected by a reader and used to infer the presence of an analyte that modulates interaction between particles and surfaces and therefore affects the trajectories. The particles may be sorted and directed toward distinct downstream ‘settled’ locations (where particles that are able to move eventually settle or collect due to the nature of the actuating forces and device geometric constraints), detectors, or surfaces that arrest the particles, to enable quantification of certain analytes. For example, two surface patterns in series, each binding to a different molecule, may be used to detect the presence of two different analytes that modulate interaction between the particles and the two surfaces, respectively, or to detect a cell that expresses two different antigens. The above approaches may be combined with each other and may be extended to more than two antigens or analytes. For example, a particle may bind to antigen A on a cell, then get deflected by a surface that binds weakly to antigen B on the cell, then get captured by a downstream surface that binds antigen C, and a fluorescent label that binds antigen D may be included and detected.

In some embodiments, the fluid sample in the device may remain constant over the entire time of the testing duration, or in some embodiments, it may be replaced at a certain determined time interval(s) in order to facilitate temporal monitoring of an analyte. For example, particles may be incubated with sample A, including the analyte, and allowed to settle on the surface, followed by subsequent analysis of the particles to determine the amount or behavior of analyte in sample A. After a predetermined time, fluid sample A may be removed from the device and replaced by sample B, which may contain a different amount of analyte. The behavior of the particles on the surface may change in response to this different sample (i.e. particles may be more or less mobile, more or less particles may become tethered to the surface, etc.). In some embodiments, the changing of fluids within the container may facilitate detection of different classes of information and/or more sensitivity of the analyte within the samples.

It should be appreciated that any suitable combination of the aforementioned multiplexing approaches, and/or any other approaches for detecting more than one analyte may be employed, as the present disclosure is not so limited. It should be appreciated that any combination of number of analytes and/or analyte types, particle populations, and target surfaces may be employed in any multiplexing application, as the present disclosure is not so limited.

The particles of the present disclosure may be formed of any suitable material compatible with the testing system and external forces employed. The particles may be composed of a material or combination of materials which interacts with the one or more external forces. For example, the particles may be formed of a paramagnetic material, such that they may be actuated through the testing sample fluid through an external magnetic field. The particles of the present disclosure may be formed of any suitable metallic (e.g., metals such as gold, tin, silver, copper, zinc, aluminum, alloys, etc.), ceramic (e.g., silica), paramagnetic, superparamagnetic (e.g., iron oxide), diamagnetic, polymeric, gel-like (e.g., hydrogels, xerogels, etc.), composite, dielectric, semiconductor, latex, combinations thereof, and/or any other suitable material. Exemplary embodiments of polymeric materials include, but are not limited by, poly (methyl methacrylate), polypropylene, polyethylene, high density polyethylene, ethylene vinyl alcohol, polyamide, polychlorotrifluoroethylene, cyclic olefin copolymer, polycarbonate, ethylene vinyl acetate, polyvinyl chloride, polyvinylidene chloride, polystyrene, thermoplastic elastomer, polyimide, polyethylene terephthalate, polyamides, aramids, expanded polytetrafluoroethylene, polyurethane, polyvinylidene difluoride, polybutyl esters, polyetheretherketone, polyolefins, fluoropolymers, combinations and/or copolymers thereof, and/or any other polymer or combination of polymers.

In some embodiments, at least one population of the particles may be formed of a material with desired optical properties. For example, the particles may be formed of a fluorophore, gold, silver, scattering, phosphorescent, quantum dot, combinations thereof, and/or any other material having optical properties. In some embodiments, the particles may include, or be decorated with, plasmonic structures. In some embodiments, the particles may have a labeled dye visible using the reader. The particles may be fluorescent under an applied excitation light in some embodiments. In other embodiments, the particles may include a release agent that changes its optical properties (e.g., color) upon exposure to an analyte. These and/or other appropriate materials exhibiting a desired optical property may enhance the readout of the testing system and subsequently, the sensitivity of the system.

The particles of the present disclosure may be any suitable size. It should be appreciated that the size of the particles may be compatible with the low cost and/or low power readers employed. For example, the particles may be large enough to be optically visible without the need for expensive magnification in some embodiments, though instances in which smaller particles are used are also contemplated. In some embodiments, the particles may have unique optical properties (e.g., fluorescence) which allow the particles to be smaller than the diffraction limit of visible light (e.g., ˜250 nm) but still distinguishable with the reader.

It should also be appreciated that the size of the particle may also be selected in accordance with the external forces used for operation of the system. In embodiments where the particles are driven by gravity or other body forces, the particles may be sufficiently sized to react to the external force in order to move through fluid and react with analyte in the testing sample. In some embodiments, the size of the particles may be determined by the viscosity of the testing sample fluid. In some embodiments, the size of the particles may be determined by the available area of the target surface. For example, if the target surface is a flat rectangular area, and the particles are generally spherical in shape, the larger the particles, the larger the number of analytes which may tether a particle to the surface based on the tangential relationship of the spheres and flat surface. The particles may be large enough to move through the fluid and settle on the target sample. However, it should be appreciated that the larger the particles, the smaller the number of particles that may be included in the device due to spatial constraints and the higher the non-specific interaction with the surface. However, if the particles are too small, they may be limited by diffusion dynamics and prolong the testing results.

In some embodiments, a population of particles may have an average transverse dimension (e.g., an average diameter) greater than or equal to approximately 300 nm, 500 nm, 1 μm, 2μ, 3 μm, 4μ, 5μ, 6 μm, 8 μm, 10 μm, 15 μm, 20 μm, and/or any other suitable size. The population of particles may have an average transverse dimension less than or equal to approximately 20 μm, 15 μm, 10 μm, 8 μm, 6 μm, 5 μm, 4 μm, 3 μm, 2 μm, 1 μm, 500 nm, 300 nm and/or any other suitable size. Combinations of the foregoing, including particles with an average transverse dimension between approximately 1 μm and 10 μm, 2 μm and 5 μm, 300 nm and 10 μm, and/or others, are also contemplated. In some embodiments, a population of particles may have average transverse dimension of approximately 4.5 μm. As noted earlier, the particle size may be dependent on a variety of factors and parameters of the testing system, such that the present disclosure is not limited by the particle size. Thus, ranges of particle sizes both greater than and less than those noted above are contemplated.

In some embodiments, the particles may be formed as a solid particle, a porous particle, a spiked particle, a core-shell particle, a nanoshell, a nanorod, microspheres, nanospheres, nanoparticles, rods, discs, stars, squares, polygonal cylinders, bar bells, combinations thereof, and/or any other suitable structure. The particles may be formed as bubbles, droplets, liposomes, vesicles, polymersomes, and/or any other particulate configuration. In some embodiments, the particles may include aggregates or composites of other particles. For example, a particle may be a nanowire-decorated sphere, a polymeric sphere with magnetic and plasmonic particles inside it, or a patterned hydrogel particle with a magnetic particle inside it. In some embodiments, the particles may be micro-robots capable of locomotion or communicating their position and/or environment to an external entity (e.g., sensor/processor). For example, bi-material micro-robots may be employed, which may swim through the testing sample fluid due to electrochemical reactions. In some embodiments, particles that have properties close to that of water, such as hydrogel particles or hydrogel-coated particles, or vesicles, or polymersomes, may be used to reduce van der Waals interactions between the particles and target surfaces.

In some embodiments, the particles of the testing systems may be spherical, cylindrical, elliptical, conical, ovoid, cubical, tube-like, flat-sided, irregular, oblate, football-shaped, discoid, slender, and polyhedral, combinations thereof, and/or any other appropriate shape. In some embodiments, the particles may include a rounded portion (e.g., hemispherical) and at least one flat surface for binding to the target surface. The particles of the present disclosure may be symmetric or asymmetric. In some embodiments, the particles may be uniform in shape, whereas in others, the particle population may include more than one shape. In some embodiments, the particles may include patterns on the surface or inside of the particles. As described previously, in embodiments where more than one analyte of interest may be present in the testing sample, the testing system may employ more than one particle population. As such, the more than one particle populations may be shaped in the same manner, but have different material properties and/or binding coatings, or may be shaped, patterned, or composed differently. It should be appreciated that any of the particles of the present disclosure may have any suitable shape or combination of shapes, compositions, or patterns as the present disclosure is not limited by particle shape or composition.

In some embodiments, the mechanical compliance of the particles may be changed to influence particle-surface interaction. For example, compliant particles may interact more easily to a target surface compared to rigid particles.

It should be appreciated that any material, size, shape, type, configuration, and/or other appropriate property of a desired particle population may be employed in the testing systems described herein as the present disclosure is not limited to any particular type of particle.

In some embodiments, a secondary fluid, different from the testing sample fluid, may be included in a system to promote separation of particles from non-target components of the sample fluid, which may include cells and cellular organelles, organic and inorganic debris, etc. In some embodiments, the secondary fluid may facilitate the separation of particles in complex fluids such as blood, which may include a variety of non-target materials in addition to the target analyte. The secondary fluid may have any suitable fluid property, such as a density or viscosity greater than, less than, or equal to that of the fluid testing sample and/or particles. The secondary fluid may also be of a lower or higher density, or a lower or higher viscosity than the sample matrix and/or the particles. For example, operating under gravity, the sample testing fluid having dispersed particles may be layered over a secondary fluid with a density that is higher than the testing sample fluid and nontarget components, but lower than the density of the particles. This arrangement may allow the particles to settle on to the surface below, while separating the particles from, and preventing the less-dense nontarget components from settling out of the sample fluid. In some embodiments, the particles or sample above the layered fluid may be actuated to promote mixing and binding of the analytes to the particles without mixing the sample with the layered fluid, after which particles may move under gravity and/or other force through the layered fluid to arrive at the target surface.

In some embodiments, the container may contain a buffer, medium, or powder such that the density of the solution resulting after addition of the sample may result in background particulates in the sample (e.g., cells) to float up, away from the target surface. Conversely, the particles may be hollow or less dense such that they float up to the target surfaces due to buoyancy forces, while background particulates may tend to settle down.

In some embodiments, the particles and the surfaces may be coated with affinity agents that have affinity for each other. The target analyte may be an enzyme that cleaves the molecules, or an analyte that competitively binds to the particles and/or surface(s), thereby decreasing the degree of adhesive interaction between the particles and the surface(s). In some embodiments, the system may include a material (e.g., an enzyme) which may disrupt the interaction between the particles and the surfaces by modifying the targeted affinity agents (e.g., through cleaving). For example, the target analyte may be an enzyme inhibitor, such that the presence of the target analyte will lead to greater adhesion between the particles and the surface compared to the case where the analyte is absent or where the analyte is present at a lower concentration. Conversely, the analyte may be a co-factor that enhances the enzyme's activity, which may then decrease adhesion between the particles and surface(s). In this way, the presence of the analyte may indirectly induce binding between the target surface and particles.

In some exemplary embodiments, the interaction between particles and target surfaces may be modulated by incorporation of additional components into the container that interact with the target analyte to result in a change in particle binding. For example, enzymes such as Cas9 are known to cleave a DNA molecule with a specific sequence when an RNA molecule of the same sequence is present. The particle and target surface may be functionalized with oligonucleotides, and an oligonucleotide that has a part that is complementary and binds to a part of the oligonucleotide on the target surface and a part that binds to a part of the oligonucleotide on the particles may be immobilized on a porous support or porous particles (to prevent interaction between the particles and the target surface) via a sequence that matches an RNA sequence of interest. Cas9 may be included in the container. If the target analyte (RNA) is present, the Cas9 may cleave and release the oligonucleotide, which, in turn, may promote binding between the particle and the target surface. In other exemplary embodiments, an antigen that facilitates binding between the particle and the target surface may be tethered inside the particles (e.g., inside porous or hydrogel particles, and/or within coatings of the particles) using a peptide tether with a certain sequence to detect an enzyme that cleaves the peptide and releases the antigens to promote binding of the particles and the target surface. In some embodiments, the target analyte may compete with an antigen included in the container such that particle binding may decrease in the presence of the analyte, analogous to competitive immunoassays. In other embodiments, an analyte may change the configuration of an aptamer or modify an affinity ligand such that it promotes binding between the particles and the target molecules.

In some embodiments, the system may include an additional material for blocking non-specific binding (e.g., albumin, casein, detergents, surfactants, etc.), and/or materials to facilitate the culture growth of target cells or organisms. In some embodiments, the system may include media which may reduce the risk of growth of certain, unwanted organisms. In some embodiments, the system may include lysis buffer to remove certain background cells. In some embodiments, the system may include lysis buffer to release the contents of certain cells for analysis. In some embodiments, the system may include an enzyme substrate for particle detection. For example, the particles may be additionally functionalized with an enzyme such as horseradish peroxidase (HRP) or luciferase, as well as a substrate the produces color, luminescence, or an electrochemically active substrate to facilitate detection of particles bound to the surface(s). In some embodiments, the system may include one or more reference analyte(s) to calibrate or provide positive controls. The additional material may include reference analyte(s) along with reference particles and reference surface(s) that can detect the reference analyte(s) to calibrate or provide positive controls. The reference analytes or particles may include fluorescence, color, magnetic, enzymatic, or other labels or behaviors (such as degrading/dissolving) to facilitate identifying them as reference analytes by a detector. The reference surfaces may be included as a patterned region or regions within or proximate to the target surfaces.

The testing systems described herein may be employed to detect any suitable biological and/or chemical analyte. In some embodiments, the testing systems may be used to measure how an analyte modulates interaction between two molecules, one coated on the particle and one on the target surface. For example, the testing systems described herein may be used to screen drug candidates. In some embodiments, the testing system may accept a fluid testing sample, which may either be a naturally occurring fluid or a solid dispersed in a liquid, such as samples which are eluted from solid supports, such as clinical specimens collected on swabs or water washes used in food preparation. In some embodiments, the testing systems of the present disclosure may test a sample from a subject (e.g., a human subject or an animal subject) and/or from an environmental space (e.g., food, seawater, etc.). Exemplary, non-limiting analytes include nucleic acids, proteins, whole organisms (viruses, bacteria, fungi, or protozoa), whole animal or plant cells, pesticides, metal ions, metabolites, protons (pH sensing), nitrate, fluoride, arsenic, endotoxins, cytokines, hormones, enzymes, peptides, drugs, or other appropriate analytes. Samples from subjects may include, but are not limited to, bodily fluids (e.g., mucus, saliva, blood, serum, plasma, amniotic fluid, sputum, urine, fecal, pus, cerebrospinal fluid, lymph, tear fluid, feces, or gastric fluid), cell scrapings (e.g., a scraping from the mouth or interior cheek), exhaled breath particles, wound discharge, tissue extracts, and culture media (e.g., a liquid in which a cell, such as a pathogen cell, has been grown). Environmental samples may include, but are not limited to, agricultural products or other foodstuffs and their extracts, rinse water from agricultural products, industrial wastewater, agricultural runoffs, drinking water, surface water, ground water, and seawater. In some embodiments, the sample may include a nasal secretion. In some embodiments, the testing systems and methods described herein may be used to detect and subsequently diagnose at least one disease or disorder caused by a virus, bacteria, fungus, protozoan, parasite, and/or cancer cell. Analytes may include whole viruses, whole cells, products of cell lysates released from the cells or viruses after lysis, antigens present in blood or other body fluids, antibodies, cytokines, or metabolites. For example, p24 antigen may be measured to diagnose HIV infection. Troponin I may be measured to detect injury to the heart. The testing systems of the present disclosure may be employed to detect more than one population of analyte in one or more testing samples in a multiplexed format. It should be appreciated that the testing systems described herein are not limited by the analyte type or number of analytes present in the testing sample.

It should be appreciated that the fluid of the testing sample may be any suitable flowable material, including, but not limited to, aqueous fluids or complex fluids such as blood. The specificity of the affinity agents employed may allow the testing systems to identify the presence of one or more target analytes even in a heterogenous mixture such as blood. It should be appreciated that the testing systems described herein are therefore not limited by the fluid of the testing sample. In some embodiments, a solid or gaseous sample may be suitably processed to extract analytes into a liquid for measurement. For example, a gaseous sample may be bubbled through a liquid to dissolve the analytes into the liquid. A soil or rock sample may be processed to extract analytes into a liquid.

The testing systems described herein may be employed for any suitable application, including, but not limited to, (1) diagnosis of infectious disease via qualitative (present/not present) or quantitative pathogen or protein sensing, (2) individual patient or mass screening for disease, condition, or compound consumption via sensing of associated cell types, metabolites, drugs, hormones, and protein markers, (3) monitoring of efficacy after therapeutic intervention for disease and tracking of disease biomarkers to investigate new treatment protocols and therapeutic agents or to inform subsequent clinical decision, (4) field testing of biological water quality through measurement of bacteria, protozoa and viral pathogens or protein markers, especially for disaster response, (5) testing for contamination by microorganisms, toxins or synthetic materials in foods, beverages, illicit and pharmaceutical drugs, and materials involved in their production (such as wash-water for leafy vegetables) for prevention of recalls and sickness, (6) rapid identification of biological national security threats such as anthrax and ricin in unknown or suspicious powders and materials, (7) testing of contaminants or toxins in air, (8) screening of drug candidates, such as inhibitors of protein-protein interactions, and (9) testing of organic or inorganic pollutants in water, such as pesticides or heavy metals. Specific non-limiting examples of applications include SARS-COV-2 diagnosis from saliva or upper respiratory swabs, HIV diagnosis, respiratory infection panels differentiating between viruses and bacteria, sepsis diagnosis, urinary tract infection diagnosis from urine, pregnancy tests, blood cell counting, antibody sensing, drug metabolites, toxin and cytokines detection, proteins markers associated with specific diseases, pathogen load after antibiotic or antiviral treatment, CD4+ T-cell monitoring in HIV treatment, fecal coliform determination, rotavirus presence in drinking water, cholera, sensing contaminants in beverages, produce, animal products, baby formulas, and ricin and anthrax sensing.

It should be appreciated that the testing systems and associated methods described herein may be safely and easily operated or conducted by untrained individuals. Unlike prior art diagnostic tests, some embodiments described herein may not require knowledge of basic laboratory techniques (e.g., fluid transfer techniques such as pipetting). Similarly, some embodiments described herein may not require expensive laboratory equipment (e.g., high resolution microscope).

In view of the above, the testing systems and related methods described herein may be useful in a wide variety of contexts. For example, in some cases, the testing systems and methods may be available over the counter for use by consumers. In such cases, untrained consumers may be able to self-administer the diagnostic test (or administer the test to friends and family members) in their own homes (or any other location of their choosing). In some cases, testing systems and related methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the testing systems and related methods may be operated or performed by the test subjects (e.g., students, teachers, patients, employees) or by designated individuals (e.g., a school nurse, a teacher, a school administrator, a receptionist). Of course, the use of the currently disclosed systems and methods by trained personnel in a laboratory environment and/or any other application is also contemplated as the disclosure is not limited to where or how the currently disclosed systems and methods are used.

In some embodiments, a testing system may include instructions to guide a user through the process of performing a diagnostic test using the testing system. The instructions may include instructions for the use, assembly, and/or storage of the testing system and any other components associated with the testing system. The instructions may be provided in any suitable form, such as written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guide a user through steps to use the testing system.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

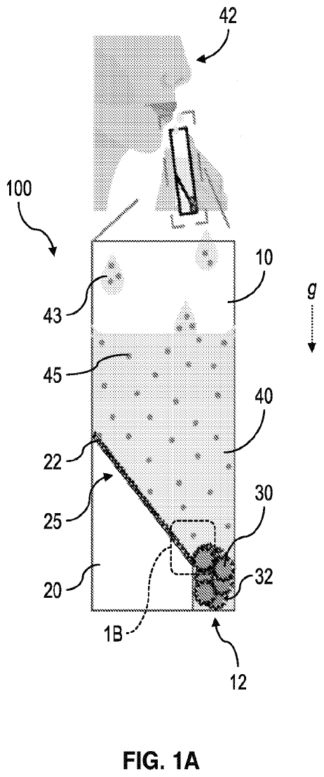

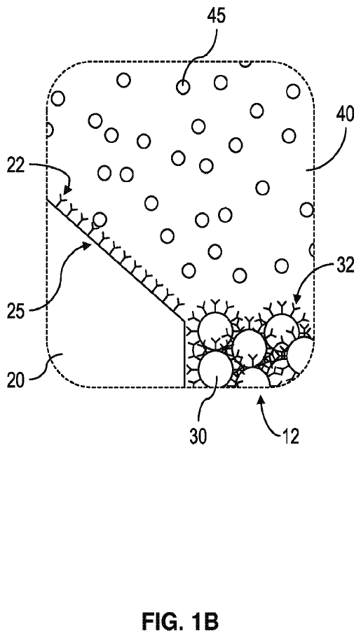

FIGS. 1A-1B show a testing system 100 according to some embodiments. The testing system 100 may be used to detect a given analyte 45 in a testing sample 43 (e.g., saliva). The analyte 45 may be dispersed in a testing sample fluid 40. As shown in FIG. 1A, in some embodiments, a subject 42 may introduce a testing sample 43 into a container 10 for testing. The container may include at least one population of particles 30 positioned in an initial location 12 of the container 10. The container may also include at least one target surface 25 for detection of the analyte 45. In some embodiments, the target surface 25 may be a surface of a body 20 within the container 10. In other embodiments, the target surface 25 may be a surface of the container itself.

In some embodiments, the container 10 may be sized similarly to a standard cuvette used in conventional detection systems. The container 10 may have a size of approximately 1 cm×1 cm×5 cm. In this way, the container may be readily manufactured with systems already in place for constructing cuvettes, and may have the added benefit of being compatible with existing optical characterization systems for spot checks. Of course, embodiments where the container is sized differently from a cuvette, either larger than or smaller than any dimension, are also contemplated. The container may hold any volume of fluid, greater than or equal to 0.1 mL, 0.5 mL, 1 mL, 2 mL, 5 mL, and/or any other suitable volume. In some embodiments, the container may hold less than or equal to 5 mL, 2 mL, 1 mL, 0.5 mL, 0.1 mL, and/or any other suitable volume. Combinations of the foregoing, including containers containing volumes between 0.1 mL and 5 mL, are also contemplated. It should be appreciated that the container may have any size, be shaped in any form (including, but not limited to polyhedral, cylindrical, or other appropriate shapes), and may hold any suitable volume of fluid, as the present disclosure is not limited by the geometry or volume capacity of the container.

FIG. 1B shows an expanded view of the surface 25 and an initial particle location 12 of the container 10 shown in FIG. 1A. As shown, in some embodiments, the surface 25 may be coated with and/or include an affinity agent, such as a binding ligand 22 which may react with/bind with analyte 45. In some embodiments, the particles 30, which may be initially arranged in an initial particle location 12 of the container, such as a well, reservoir, or other structure, may also be coated with and/or include an affinity agent, such as a binding ligand 32. As described previously, the particles 30 may be urged out of their initial location with an external force and subsequently moved through fluid 40 in order to bind to one or more analyte 40. The analyte-laden particles may then settle on the target surface 25. In some instances, the analyte bound to the particle 30 may also bind to the target surface 25, such that the particle 30 may be bound to the surface 25 through the analyte 45.

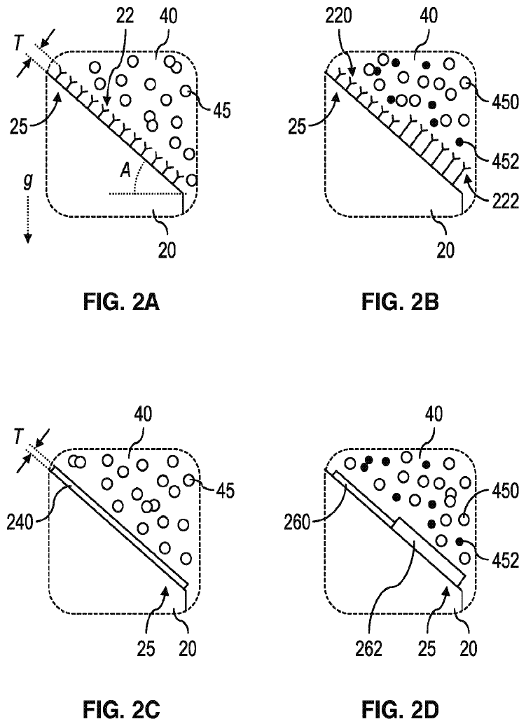

FIGS. 2A-2D show various embodiments of a target surface 25. As will be described in greater detail below, in some embodiments, the target surface 25 may be angled at an angle A relative to the external force g, which may be gravity. The angle may serve to disengage a population of the non-specifically bound particles from the target surface. The angled target surface may enable particles to slide/roll along the surface and subsequently off of the surface if the non-specific binding force between the analyte and the target surface is less than the external applied force. In this way, the angled target surface, which may be understood to apply a force tangential to the target surface to the bound particles, may serve a secondary function of discouraging non-specifically bound particles from binding on the surface.

The angle A may be any suitable angle, including greater than or equal to 10°, 20°, 30°, 40°, 50°, 60°, 70°, 80°, 90°, and/or any other suitable angle. The angle A may also be less than or equal to 90°, 80°, 70°, 60°, 50°, 40°, 30°, 20°, 10°, and/or any other suitable angle. Combinations of the foregoing, including angles A between 0° and 90°, are also contemplated. In some embodiments, angle A may be between 30° and 85°. In some embodiments, the target surface 25 may be normal relative to the external force, such that angle A may be 0°. It should be appreciated that the angle of the target surface relative to the external force may be determined by a variety of other factors, including dynamic fluid properties of the testing sample fluid, binding energies of the surface affinity agents of the particles and the target surface, among others. As such, the present disclosure is not limited by the angle of the target surface relative to the external force.

It should be appreciated that in some embodiments, the target surface may have a non-planar configuration, which may maximize its analyte binding area. However, planar target surfaces may have the advantage of being more readily imaged using the reader. Of course, the target surfaces of the present disclosure may have any planar or non-planar configuration, as the present disclosure is not so limited. In some embodiments, the target surfaces of the present disclosure may include topographical patterns, including, but not limited to, undulating ridges or channels, which further aid in reducing non-specific interactions or directing non-specifically bound particles off of the target surface.

It should be appreciated that target surfaces may be continuous or discrete. In some embodiments, the target surfaces have a topography to collect particles that move on the surfaces. For example, the target surfaces may have wells or troughs to collect particles that fall outside the wells or troughs, but move on the surface randomly (as in Brownian motion) or under the influence of some force. In some embodiments, the target surfaces may be formed of suspended fibers of circular or non-circular cross-sections, spheres, spheroids, or ribbons.

In some embodiments, a body 20 including the functionalized target surface 25 formed thereon, shown in FIGS. 1A-2D, may be formed separately from the container 10, whereas in other embodiments, the body may be monolithically formed as part of the container. In some embodiments, the body may be pre-functionalized with the surface ligand 22, and then positioned within the container, such that only the surfaces of the body may specifically bind to the analyte. In other embodiments, the target surface may be functionalized within the container 10. It should be appreciated that the target surface or surfaces may be realized in any suitable fashion, as the present disclosure is not so limited.

FIG. 2B shows a target surface 25 of a multiplexing testing system having two portions, each covered by a respective one of binding ligands 220 and 222 for binding two different analytes 450 and 452, respectively. The analytes 450 and 452 may each be bound to different types of particles (not shown), which may exhibit different optical properties (e.g., fluorescence). Accordingly, a sequence of images captured from the target surface may differentiate between the two analyte populations. It should be appreciated that although both types of ligands 220 and 222 are shown to be on the same target surface 25, embodiments in which have more than one target surface, each having a uniform coating of ligand 220 or 222, are also contemplated. Thus, images of two target surfaces may be collected in order to determine the presence and concentration of the two analyte populations. In some embodiments, as shown in FIG. 2B, each ligand type may be arranged in a patch, such that the resultant images collected from the target surface may show a first portion of the surface covered in a first population of particles, and a second portion of the surface covered in the second population. Image analysis (e.g., digital data analysis, including image analysis algorithms, statistical methods, or machine learning) may be applied to the observations of particle motions in order to distinguish the two analyte populations based on their position and motion on the target surface. It should be appreciated that any suitable arrangement of the various ligands may be employed (e.g., ligands interspersed, ligands arranged in a pattern, etc.), as the present disclosure is not so limited.

In some embodiments, the binding ligands 220 and 222 may be sufficiently different such that each ligand may bind to its targeted analyte following binding to a particle. The binding energies of the various binding ligands used herein may have varying affinities and cross-reactivities for multiplexing applications. In some embodiments, analyte testing systems may be used to identify similar classes of analytes that may have differential binding to a set of antibodies (e.g., zika virus, dengue, etc.). It should be appreciated that the height difference shown between ligands 220 and 222 is demonstrative only and does not necessarily indicate differences in ligand height.

FIG. 2C depicts a target surface which may be coated with an affinity agent in the form of a binding coating 240 for binding to the analyte 45 while reducing the surface van der Waals forces in order to limit non-specific binding. In some embodiments, as represented by FIG. 2D, the target surface may be coated with and/or include more than one binding coating 260 and 262, for binding to two analyte populations 450 and 452. Similar to the arrangement described relative to FIG. 2B, the two binding coatings may be arranged in any suitable fashion to facilitate analysis of the two analyte populations when images of the target surface are collected. As described previously, in some embodiments, more than one target surface, each coated with a binding coating, may alternatively be employed for multiplexing applications. It should be appreciated that the differences in coating thickness shown are demonstrative only and not necessarily representative of the coating thicknesses.

The testing systems of the present disclosure may employ any suitable affinity agent. In some embodiments, the affinity agent may be a binding ligand, coated on and/or included in either the target surface(s) and/or the particles. The target surfaces may be functionalized or coated to react with a targeted moiety of the analyte, and may, in some embodiments, exhibit antifouling properties which may limit non-specific binding. In some embodiments, the binding ligand coatings may reduce the van der Waals forces between the target surface and the particles in order to reduce non-specific binding. In some embodiments, binding ligands may be attached to the target surfaces on top of the antifouling coatings, the target surfaces may be coated with a mixture of affinity ligands and antifouling molecules, and/or combinations thereof may be used.

The binding ligands may include, but are not limited to, antibodies, monoclonal or polyclonal antibodies (or combinations thereof), oligonucleotides, aptamers, ion-mediated functional groups, polyethylene glycol (PEG) and related copolymers, zwitterionic coatings, hydrogels, polymer brushes, albumin, click chemistries, enzymes, peptides, carbohydrates, glycoproteins, major histocompatibility complex, receptors, drug molecules, thiols, carboxyl groups, silanes, ion chelators, nanobodies, antigens, viruses, live or dead cells, biofilms, hydrogels, porous materials, xerogels, polymer brushes, macromolecular networks, polymer matrices formed of synthetic and/or natural materials, combinations thereof, and/or any other suitable coating material.

In some embodiments, the affinity ligands may be located on particles that are attached to the target surfaces in order to distinguish between non-specific and specific binding, or, in some embodiments, they may be used for multiplexing through differences in color, shape, pattern, magnetic properties, or other properties or features of the ligand-coated particles, with distinguishable particles being coated with different ligands.

In some embodiments, the binding ligands may coat the target surface and/or particles at any suitable surface density, including greater than or equal to 1, 10, 100, 1000, 10,000, 100,000 μm⁻², and/or any other surface density. In some embodiments, the surface density of the ligands may also be less than or equal to 100,000, 10,000, 1000, 100, 10, 1, and/or any other surface density. Combinations of the foregoing, including a surface density of binding ligands between 1 and 100,000 bind ligands per square micron, as well as ranges above and below the aforementioned, are also contemplated. In some embodiments, the ligands may be clustered in patches on the target surface and/or particles, whereas in others, the ligands may be substantially uniform along the target surface and/or particles.

It should be appreciated that the particles may be coated with and/or include a first binding ligand type to bind to a population of analyte, and the target surface may be coated with and/or include a second binding ligand type to bind to the analyte bound to the particles. In some embodiments, the first and second binding ligand types may react with different moieties of the analyte. For example, pairs of antibodies that are often used for lateral flow assays or ELISAs that target different epitopes of the analyte may be used on the particles and target surface, respectively. Thus, in some embodiments, the target surface and the particles may be coated with the same binding ligand, whereas in other embodiments, the target surface and the particles may be coated with different binding ligands. In multiplexing embodiments, each target surface and/or particle type may be coated with the same and/or different binding ligand. It should be appreciated that the present disclosure is not limited by the type of binding ligands on the target surface or on the particles. In some embodiments, a particle and/or target surface may be coated with one or more binding ligands.

In some embodiments, the affinity agent coating may include one or more layers formed of the same and/or different materials. In some embodiments, the target surface and the particles may be coated with the same binding coating, whereas in other embodiments, the target surface and the particles may be coated with different binding coatings. In multiplexing embodiments, each target surface and/or particle type may be coated with the same and/or different binding coating. It should be appreciated that the present disclosure is not limited by the type of binding coating(s) on the target surface or on the particles. In some embodiments, a particle and/or target surface may be coated with one or more binding coatings.

The affinity agents of the present disclosure may be deposited on the target surface and/or particles in any suitable manner. In some embodiments, thin films of affinity agents may be formed using any suitable technique, including, but not limited to, using chemical vapor deposition, physical vapor deposition, layer-by-layer, spin-coating, self-assembly, dip-coating, atomic layer deposition, Langmuir-Blodgett, physical vapor position, evaporative (e.g., electron beam evaporation techniques, thermal evaporation techniques), sputtering, lithography (e.g., photolithography, electron beam lithography, etc.), combinations thereof, and/or any other techniques.

In some embodiments, the affinity agents may include hydrogels, polymers, and/or other means to enhance binding and/or mitigate non-specific binding. The particles or surfaces coated with the affinity agents may be corrugated or roughened to modulate binding or to minimize non-specific binding.

It should be appreciated that any of the particles and surfaces described herein may be coated with any of the affinity agents described herein, as well as combinations of affinity agents (e.g., ligands and coating), as the present disclosure is not limited by the binding chemistries of the various components of the testing system.

As shown in FIGS. 2A and 2C, in some embodiments, the binding coating 240 may have a thickness T of any suitable size, including, but not limited to, greater than or equal to 0.5 nm, 1 nm, 2 nm, 5 nm, 10 nm, 20 nm, 50 nm, 100 nm, 200 nm, 300 nm, 500 nm, 700 nm, 1 μm, 2 μm, 5 μm, 10 μm, 20 μm and/or any other suitable thickness. The binding coating thickness may also be less than or equal to 20 μm, 10 μm, 5 μm, 2 μm, 1 μm, 700 nm, 500 nm, 300 nm, 200 nm 100 nm, 50 nm, 20 nm, 10 nm, 5 nm, 2 nm, 1 nm, 0.5 nm, and/or any other suitable thickness. Combinations of the foregoing, including binding coating thicknesses between 0.5 nm and 10 μm, 0.5 nm to 10 nm, 10 nm to 100 nm, 100 nm to 1 μm, 1 μm to 10 μm, combinations thereof, and/or any other suitable range of coating thicknesses are also contemplated. In some embodiments, the binding coating thickness may be between 100 nm and 1 μm. In some embodiments, the binding coatings may be formed of self-assembled monolayers. It should be appreciated that any of the target surfaces and/or particles described herein may be coated with and/or include binding coatings of any suitable thicknesses, including those greater than, equal to, or less than described above.

It should be appreciated that although multiplexing embodiments of the target surface is shown to interact with two analyte populations in FIGS. 2B and 2D, the testing systems of the present disclosure may be employed to detect any suitable number of analytes in parallel within one container, including, but not limited to, greater than or equal to one population, two population, three population, five populations, ten populations, and/or any other suitable number of analyte populations.

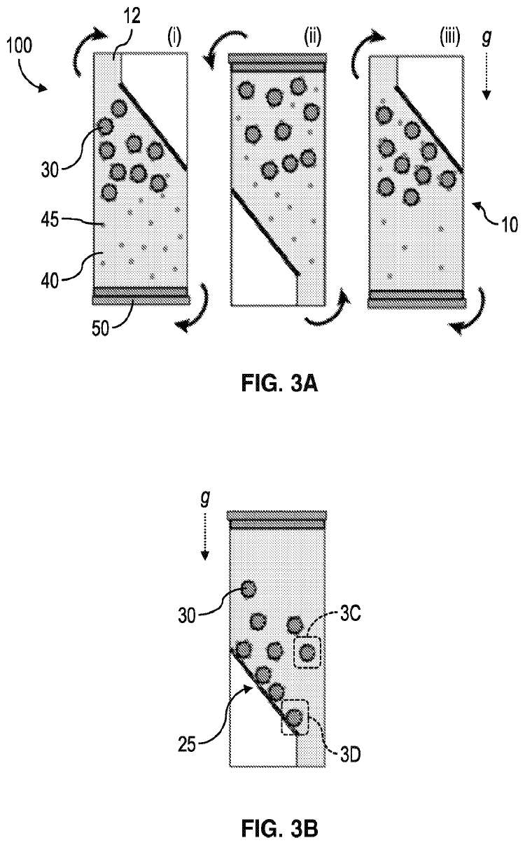

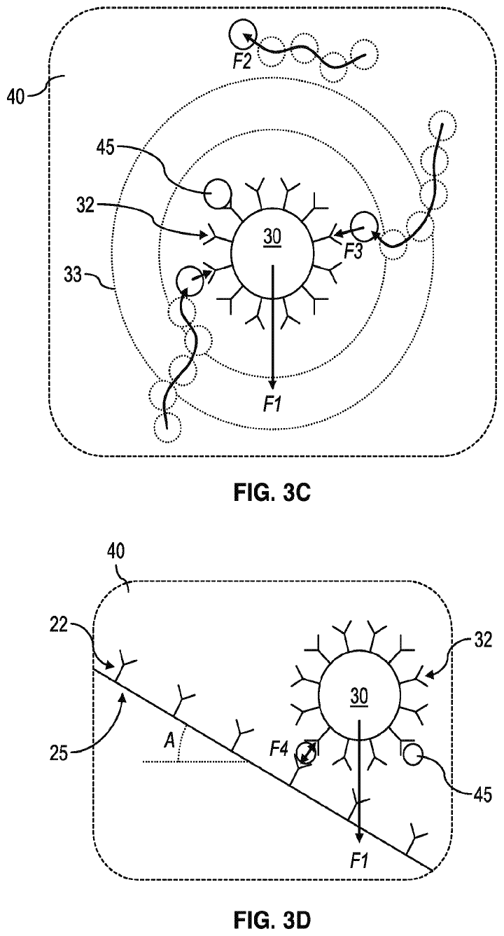

FIGS. 3A-3B depict an exemplary embodiment of a testing system 100 being mechanically agitated in order to drive particles 30 through fluid 40 of a testing sample. The container 10 may be sealed with a closure device 50 to limit fluid leakage out of the container. Upon agitation, as shown in FIGS. 3A(i)-(iii), the particles 30 may react with or bind with analyte 45 dispersed within the fluid. In some embodiments, the container 10 may be agitated relative to an external force g, which may be gravity. The particle properties (e.g., size, density, material composition, reaction to external force, etc.) may be selected to allow the particles to move through the fluid 40, such that the particles may be exposed to a greater population of analyte than if the particles were diffusion-limited. Following a short period of time, which may be predetermined based on the dynamic parameters of the fluid and particles and the binding dynamics of the particles and analyte, the particles may be directed toward a target surface 25, as shown in FIG. 3B. In some embodiments represented by FIGS. 3A-3B, the target surface 25 may be angled relative to the external force, such that particles may begin to fall through the fluid 40 toward the target surface, and a population of the non-specifically bound particles may roll off of the target surface 25. As will be described in detail below, in some embodiments, vibrational forces may be optionally applied to the testing system. In this way, the external force (e.g., gravity) may serve to both urge the particles through the fluid to react with analyte present in the fluid, and also to direct the particles to the target surface, while reducing the number of non-specifically bound particles on the target surface.

FIGS. 3C-3D depict the various forces at play for two different particles from FIG. 3B. In FIG. 3C, particle 30 is dispersed in fluid 40, with several analytes 45 nearby. The particle 30 itself may experience a first settling force F1, which may be a result of an external force directing the particle either through the fluid or toward the target surface. For example, if the external force is gravity, the force F1 may be the gravitational pull on the body of particle 30. Each analyte 45 may move through the fluid 40 with a diffusive force F2. It should be appreciated that the analytes 45 may be too small to be affected by the external force, and/or may not be formed of a material which may react with the external force. Thus, each analyte 45 may move through the fluid irrespective of the applied force. In some instances, as shown, the analyte 45 may move past a boundary layer 33 of the particle 30, which may expose the analyte 45 to a binding ligand 32. The binding energy between the ligand 32 and analyte 45, as indicated by force F3, may actively drive the analyte 45 toward the particle 30. In some embodiments, the binding force F3 between the binding ligand 32 and the analyte 45 may be strong enough such that the analyte 45 may remain bound to the particle 30 while it settles on a target surface of the container.

FIG. 3D depicts another particle 30 which may be specifically bound to the target surface 25 of the container. The particle 30 may continue to experience the first force F1 (e.g., gravitational force) relative to the external applied force. If the target surface is angled, as is shown in FIG. 3D, the particle 30 may be urged further down the ramp of the target surface in order to minimize its potential energy. However, as shown, the gravitational force F1 may be countered by a binding force F4 between the particle 30 and binding ligands 22 on the target surface 25, mediated by one or more analytes 45. The ligand-analyte-particle binding force, which may be considered specific binding of the particle to the surface, may be strong enough to prevent the particle from rolling off the surface in accordance to force F1. In this way, the particle may remain on the target surface, and its dynamic movement may be evaluated to quantify the concentration of analyte in the testing sample.

In some embodiments, the process of analyte capture by the ligand functionalized particles exposed to gravity as the external force may be modeled as two resistances in series: (1) analyte mass transfer to the surface of the particle and (2) kinetics of analyte binding to ligands on the particle surface. In embodiments where the analyte is a virion and the particles are antibody-functionalized microspheres, the second resistance may represent the kinetics of virion surface protein binding to antibodies on the sphere surface. In the regime in which the surface of the particles is not saturated with analyte (e.g., when the available binding area on the particles is greater than the number of analytes in solution), the concentration of analyte in solution may exponentially decay with time as follows:

$$\frac{C ( t )}{{C}_{0}} = exp [ - \frac{{N}_{p} ( 4 π {r}_{p}^{2} ) t}{{V}_{s} {R}_{total}} ]$$

In the equation above, C(t) is the concentration of analyte in the testing sample solution (number of analyte per unit volume) at time t, C₀ is the initial concentration of analyte in solution, Nₚ is the number of particles, rₚ is the radius of the particle, Vₛ is the volume of the sample, and Rₜₒₜₐₗ is the total resistance that includes the mass transfer and reaction resistances, which may be equal to the sum of the analyte mass transfer resistance Rₘₐₛₛ and the analyte binding resistance R_{react} as follows:

$${R}_{total} = {R}_{mass} + {R}_{react}$$

In the equation above, Rₘₐₛₛ may be determined by the movement of the analyte through the diffusion/boundary layer on the particle surface as follows:

$${R}_{mass} = \frac{δ}{D}$$

Where & is the layer thickness and D is the analyte diffusivity. Layer thickness δ may be estimated by balancing the analyte diffusion time and the particle sedimentation time, which in turn depend on the particle diameter and sedimentation velocity, uₚ (m/s) as follows

$$δ ∼ {( \frac{4 {r}_{p} D}{{u}_{p}} )}^{1 / 3}$$

For low Reynolds number conditions, uₚ may be found by balancing the gravitational force, the buoyant force and the Stokes' drag force on the particle as follows:

$${u}_{p} = \frac{2}{9} \frac{( {ρ}_{p} - {ρ}_{f} ) {gr}_{p}^{2}}{{μ}_{f}}$$

Where ρₚ is the density of the particle, ρ_{f} is the density of the testing sample fluid, g is the acceleration of gravity, and μ_{f} is the dynamic viscosity of the fluid. In some embodiments, the analyte (e.g., virion) may be modeled as a soluble antigen, such that known binding on-rates between the analyte and particle (k_{3D}^{on}) may be used to predict the binding to a target surface with ligand density L₀ (mole/m²) as follows:

$${R}_{react} = \frac{1}{{k}_{on}^{3 D} {L}_{0}}$$

Resulting in:

$${R}_{total} = \frac{{k}_{on}^{3 D} {L}_{0} δ + D}{{k}_{on}^{3 D} {L}_{0} D}$$

Such a model may use experimentally determined on-rates for the target surface ligand and particle ligand but may not account for the particulate nature of the analyte.

In some embodiments, the analyte may be modeled as a nanoparticle. Experimentally determined linear correlations between target surface ligand density and particle surface ligand density may be used to predict an association rate of the analyte binding to the target surface (KA), which may be used as the reaction resistance:

$${R}_{react} = \frac{1}{{k}_{A}}$$

Resulting in:

$${R}_{total} = \frac{{k}_{A} δ + D}{{k}_{A} D}$$

Such a model considers the particulate nature of the analyte and the geometrical constraints introduced during binding. In embodiments where the analyte is large compared to the above convection-diffusion based estimate of δ, as may be the case for capture of bacteria and mammalian cells, hydrodynamic considerations may need to be considered to estimate the capture rate. A simple model considers a thin-shell volume of solution around each particle, on the order of the critical length scale of the analyte (l_{analyte}), to estimate the volume sampled per particle per time, \dot{V} as follows:

\dot{V}=πrₚuₚl_{analyte}

In some embodiments, the volume sampled per particle per time, \dot{V}, may be used in the exponential decay equation in place of 4πrₚ²/Rₜₒₜ to estimate the rate of analyte uptake.

In embodiments with target surfaces arranged in an angled format (see, for example, FIG. 2A), the rolling and tethering of the analyte-particle complexes may be modeled as a sphere with surface asperities of the size of the analyte. To estimate the translational velocity (u) on an incline of angle A, one can use a model for no-slip rolling of particles on an incline in low Reynolds number conditions as follows:

$$u = \frac{{u}_{p} sin ( θ )}{{F}_{t} + {F}_{r} + \frac{3}{4} ( {T}_{t} + {T}_{r} )}$$

Where uₚ is the sedimentation velocity of the particle, defined previously. The nondimensional factors in the denominator, Fₜ, Fᵣ, Tₜ and Tᵣ, may be empirically determined resistance functions which relate hydrodynamic force and torque to the particle's motion based on the particle's surface roughness due to asperities. They take the following forms:

$${F}_{t} ∼ - \frac{8}{15} ln ( ξ ) + 0.9588 \qquad {T}_{t} ∼ \frac{1}{10} ln ( ξ ) + 0.1895$$

$${F}_{r} ∼ \frac{2}{15} ln ( ξ ) + 0.2526 \qquad {T}_{r} ∼ - \frac{2}{5} ln ( ξ ) + 0.3817$$

Where ξ is the nondimensional roughness scale, equal to the average size of the surface asperities divided by the radius of the particle. Dividing the length of the inclined target surface by the translational velocity u allows an estimate for the time required for a particle to roll down the complete length of the target surface. However, as noted previously, in some embodiments, the particles may be tethered to the target surface through specific ligand-analyte-ligand binding of the particle. This tethering may be modeled by considering a single target surface ligand-analyte force (although there will likely be multiple bonds) and moment balance on the particles. This static balance may lead to an estimation of the force Fₜₑₜₕₑᵣ required to stably hold the particle to the inclined target surface at angle A as follows:

$${F}_{tether} = \frac{( {ρ}_{p} - {ρ}_{f} ) {V}_{p} g sin ( A )}{{[ 1 - \frac{{r}_{p}^{2}}{{( l + {r}_{p} )}^{2}} ]}^{\frac{1}{2}}}$$

Where Vₚ is the volume of the particle, and l is the length of the tether. Comparing Fₜₑₜₕₑᵣ to experimentally determined rupture forces between ligands and analytes may help determine the particle radius and material composition, as well as properties of the target surface (e.g., incline), in order to retain specifically bound particles on the target surface.

It should be appreciated that the analysis outlined above is for demonstration purposes only, and other methods of determining analyte concentration and system optimization may also be employed.

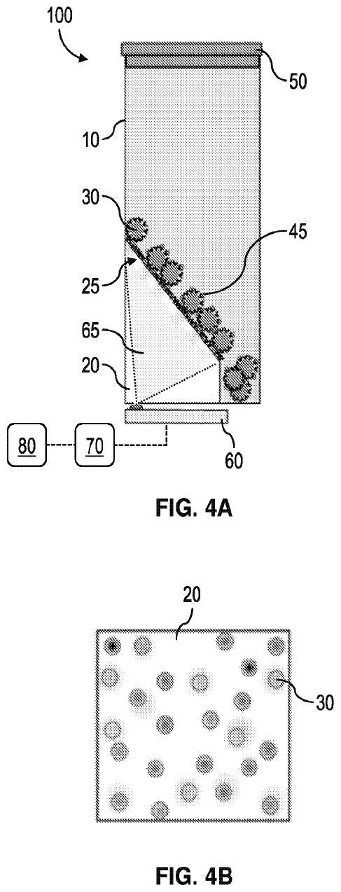

FIGS. 4A-4B depict an exemplary method of determining analyte concentration within a test sample. As described in relation to FIGS. 3A-3B, following agitation and rest, a population of particles 30 may be settled on a target surface 25. A subset of these particles may be bound to an analyte from the test sample, and subsequently bound to an affinity agent on the surface 25. The remaining particles may be nonspecifically bound to the surface. As described previously, in some embodiments, a reader may be used to capture a sequence of images from the target surface to evaluate the dynamic movement (e.g., displacement, velocity, or Brownian motion) of each particle. The movement may then be analyzed in order to identify information about analyte in the test sample. For example, Brownian motion of the particles may be calculated to determine whether or not a particle is specifically bound to the surface (e.g., more motion may suggest the particle is nonspecifically bound, not tethered by the analyte-ligand connection).

Accordingly, a testing system may employ a reader 60, which may be directly compatible with the container in some embodiments, reducing the number of operational steps, in order to image the target surface. In some embodiments, as shown in FIG. 4A, the reader 60 may include a light source for shining incident light 65 on to the target surface 45. The light may illuminate and/or interact with the particles 30 for visualization. In some embodiments, the reader 60 may include an optical digital microscope and a camera or detector, but other embodiments of the reader 60 may also be employed.

In some embodiments, the reader may apply vibration, acoustic, ultrasonic, magnetic actuation, combinations thereof, and/or other forms of actuation to mix the particles in the sample and enhance their binding with analytes, to move the particles across the target surfaces, and/or to reduce non-specific binding of particles with the target surfaces. In some embodiments, the reader may set, control, or change the orientation of the device with respect to the direction of gravitational acceleration.

In some embodiments, the body 20 may be transparent to the incident light 65, such that the reader 60 may be able to visualize the target surface with limited losses as it travels through the body 20. The body 20 may be formed of any suitable material, dependent on the light 65, including non-limiting examples of silica, transparent polymers, and/or any other suitable material or combinations of materials.

In some embodiments, the reader 60 may collect a sequence of images (see exemplary image showing particles 30 in FIG. 4B) from the target surface, storing said sequence in a non-transitory computer readable memory 70, as shown in FIG. 4A. Particles 30 settled on the target surface may be individually identified (see FIG. 4B) and tracked through the sequence of images. In some embodiments, a processor 80, connected to the reader 60 may facilitate the tracking and motion determination of the particles in order to quantify the concentration of analyte in the test solution, as described previously.

It should be appreciated that any suitable method may be employed to quantify number of and/or motion of particles settled on a target surface, including optical (e.g., brightfield, fluorescent, augmented by side scattering or grazing angle reflection), electric or electrochemical (e.g., enzyme or otherwise catalyzed), acoustic (e.g., surface acoustic waves), magnetic, spectroscopic (e.g., surface enhanced Raman spectroscopy (SERS)), combinations thereof, and/or any other suitable technique.

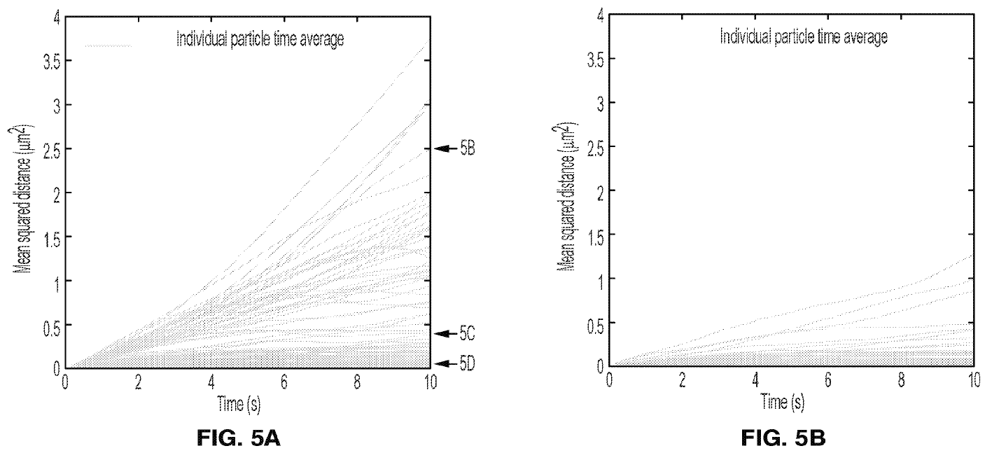

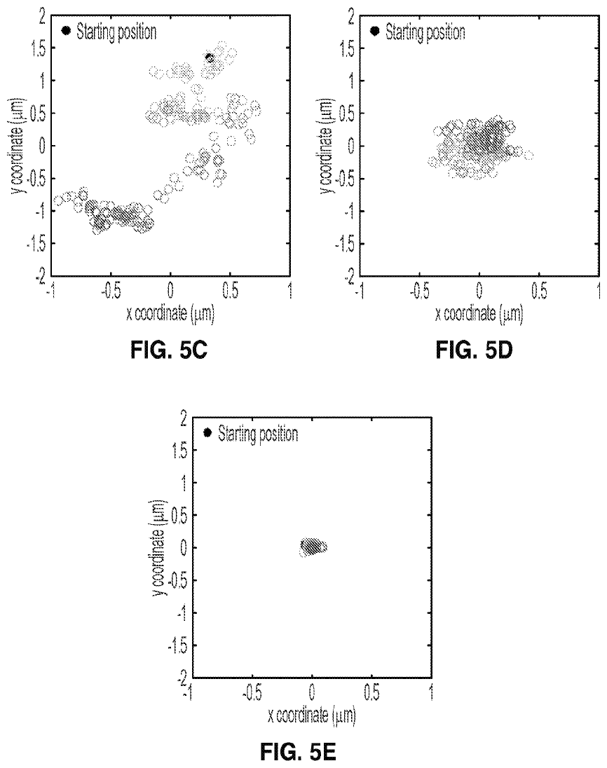

FIGS. 5A-5E show exemplary measurements of mean square distance measurements (MSD) and trajectories of individual particles on a target surface. FIG. 5A shows the MSD of a population of particles on a target surface illustrating the range of particle motions that are possible due to particle binding status. In this control condition, particles were incubated with 0 ng/ml of analyte and allowed to settle on to a surface of an antifouling polymer-coated glass slide. Particles were monitored via video microscopy and each individual particle's MSD was quantified over the recording period by analyzing bead positions in each video frame. The control condition shown in FIG. 5A illustrates the degree of nonspecific binding that occurs between particles and the surface. FIGS. 5B-5D show exemplary individual particle trajectories from FIG. 5A, as indicated. In particular, FIG. 5B shows the MSD of a freely-diffusing particle, FIG. 5C shows the MSD of a weakly bound/tethered particle, and FIG. 5D shows the MSD of a strongly bound particle. In contrast, FIG. 5E shows the MSD of a particle incubated with 1 ng/ml of analyte, illustrating that there are less freely diffusing particles due to specific interactions between particle-bound analyte and the tethering target surface. The differences in particle motions between conditions incubated with different concentrations of analyte, as illustrated in FIGS. 5B-5E may provide the basis for differentiating and quantifying the amount of analyte in the original sample.

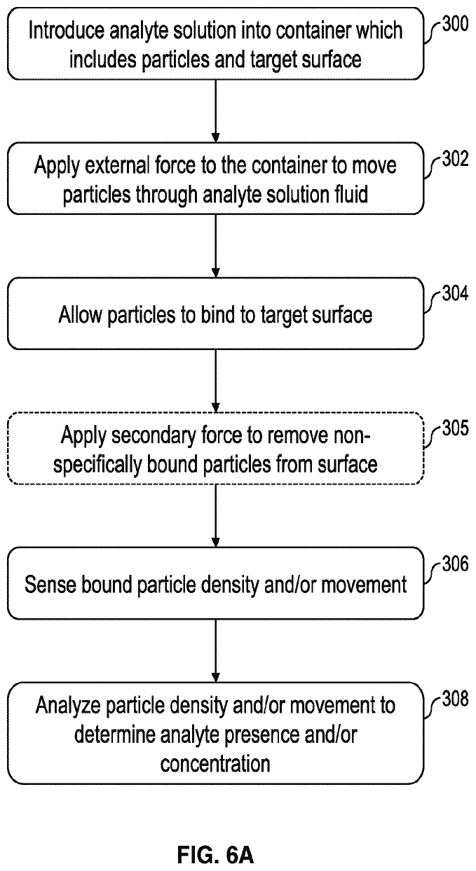

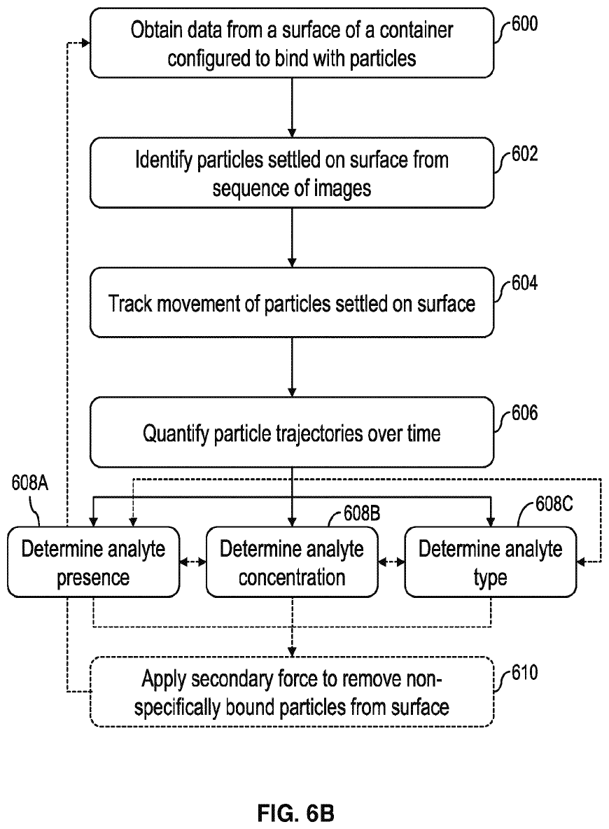

FIG. 6A shows a procedural flowchart of a method of analyte detection using the testing systems described herein. In block 300, a user may introduce analyte solution into a container, which may already house at least one population of particles and at least one target surface. In some embodiments, the user may need to introduce both the analyte solution and the particles into the container. In other embodiments, the container may be prepopulated with the analyte solution, and the user may need to introduce the particles into the container. In other embodiments, the container may be prepopulated with a solution such as a buffer solution, buffer solution with blocking agents to reduce non-specific binding, a solution containing particles, or combinations thereof. In some cases, the afore-mentioned components may be contained in the form of a lyophilized powder, capsules, or coatings that are dissolved into a solution or sample. The container may then be capped and, as shown in block 302, agitated or otherwise exposed to an external force (e.g., gravitational or magnetic, among others) in order to urge the particles to move through the fluid, being exposed to analyte in the test solution. In some embodiments, the particles may include and/or be coated with a binding ligand or coating which may allow the particles to bind one or more analytes. The analyte laden particles may then be directed to a target surface, as shown in block 304 by an external force (e.g., gravity). In some embodiments, as represented by block 305, a secondary force may optionally be applied to the container to remove non-specifically bound particles from the target surface. It should be appreciated that the magnitude of the secondary force may be lower than that required to overcome the binding energy between ligand-analyte-particle, such that the secondary force may not be strong enough to dislodge specifically bound particles. As described previously, the target surface may be interrogated using a reader (and/or any other suitable device which may collect information about particles bound (both specifically and nonspecifically) to the target surface. In some embodiments, the collected information may include sensed particle density and/or movement on the target surface, as shown in block 306. Data (e.g., images) acquired in block 306 may subsequently be analyzed in order to quantify analyte presence and/or concentration in the test sample, as shown in block 308. For example, in some embodiments, the Brownian motion of the bound particles may be evaluated from a sequence of images captured by the reader.

FIG. 6B shows a procedural flowchart for determining information about the testing sample through target surface sensing as described in relation to blocks 306 and 308 of FIG. 6A for embodiments in which images are obtained of the target surface. In block 600, the system may first obtain a sequence of images of the target surface having a population of bound particles. In some cases, the particles may be bound specifically through a ligand-analyte-particle interaction, whereas in other cases, the particles may be nonspecifically bound through surface forces such as electrostatic or van der Waals forces. Steps represented by blocks 602-610 may help distinguish the specifically bound particles from the nonspecifically bound ones, providing information about the testing sample composition. In block 602, one or more particles on the target surface may be identified in the sequence of images captured in block 600. The magnitude of the secondary force may be changed with time. In some exemplary embodiments, the magnitude of the secondary force may be increased each time the loop is executed. It should be appreciated that the exertion of the secondary force may or may not be paused when the particles are sensed. A representative image, with particles identified with boundary lines, is shown in FIG. 6B.

In some embodiments, the particles bound (either specifically or nonspecifically) to the target surface may be identified using any suitable algorithm, including, but not limited to, edge detection, contrast, thresholding, fluorescence thresholding, and/or any other suitable algorithm. Accordingly, it should be understood that any conventional particle identification and tracking algorithms may be used as the disclosure is not limited to how the particles are identified and/or tracked in individual images and/or sequences of images.

It should be appreciated that any suitable system to collect and analyze data related to particles bound (specifically and nonspecifically) on the target surface may be employed, including, but not limited to readers such as camera/microscope assemblies with data collection and image processing abilities.

Once particles are identified, their movement can be tracked over time through the sequence of images (or any other sequence of collected data), as shown in block 604. This tracking information may be used to quantify the particle trajectory (and/or Brownian motion) over time, as shown in block 606. Any suitable method for quantifying particle trajectory may be employed, such as mean squared distance calculations, displacement with time, velocity, fluctuations in velocity, fluctuations in displacement, dependence of mean square displacement with time, and/or other appropriate methods for tracking movement of identified particles within the sequence of images. In some embodiments, such measurements may determine at least one of the testing sample properties shown in blocks 608A-C, including analyte presence, analyte concentration, and/or analyte type (e.g., in multiplexing applications, when the testing sample includes more than one analyte population). In some embodiments, a greater magnitude of Brownian motion may indicate that the particle is nonspecifically bound to the surface, and not tethered by the strong ligand-analyte-particle bond. Thus, the steps outlined in blocks 600-608C may help distinguish between specifically and nonspecifically bound particles. In some embodiments, determining one testing sample property (e.g., concentration) may provide information about the others (e.g., presence). As described previously, in some embodiments, a secondary force may optionally be applied to the container to help dislodge a portion of the nonspecifically bound particles, as shown in block 610. The imaging and analysis process, blocks 600-610, may optionally be repeated in order to determine testing sample properties more accurately.

It should be appreciated that the testing systems described herein may include one or more processors and associated non-transitory computer readable memory. The non-transitory computer readable memory may include processor executable instructions that when executed by the one or more processors cause the testing system to perform any of the methods disclosed herein, including, but not limited to the data (e.g., image) analysis processes.

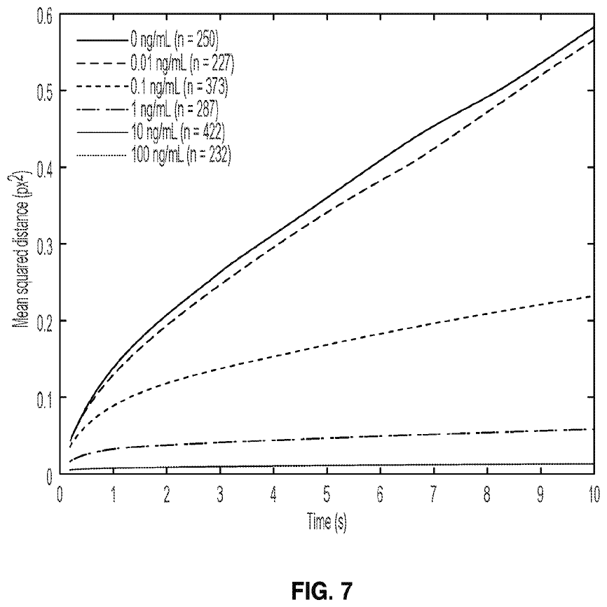

FIG. 7 shows exemplary Brownian motion of a population of particles settled on to a target surface following incubation with analyte. The particles are polymer coated superparamagnetic particles with a diameter of 4.5 μm incubated with a prototypical analyte at 0-100 ng/mL and allowed to settle onto an antifouling polymer-coated glass slide. Using brightfield microscopy of a reader, the motion of the settled particles is monitored and recorded over a 40 second time interval. Analysis of particle positions in each video frame may allow for the movement of each individual particle to be quantified and the resulting average motion of a population of particles (ensemble average) may be used to determine the concentration of the analyte originally incubated with the particles. It should be appreciated that the lower the concentration of the analyte in the original solution, the less analyte that binds to the particles and the less particles that may be specifically tethered to the surface, resulting in greater magnitude of particle motion with decreasing analyte concentration. In some embodiments, the surface may be inverted prior to video monitoring of Brownian motion to aid in removing non-specifically bound particles from the surface.

The movement of individual particles from FIGS. 5A-5E and FIG. 7 was quantified via mean squared distance (MSD), a measure of the distance the particle has moved after a specific time interval. For each single particle in the experiment, analysis of the particle in each video frame yields a set of discrete two-dimensional coordinates, which describe the particle's position over the entire observation period as follows:

r(t)=(x(t),y(t))

Where t is time. For a specified lag time t, or time between observations, the time-averaged MSD for a single particle may be calculated as follows:

$${MSD}_{x} ( τ ) = \frac{1}{{N}_{i}} {\sum }_{i = 1}^{{N}_{i}} {[ ( x ( {t}_{i} + τ ) - x ( {t}_{i} ) ]}^{2}$$

$${MSD}_{y} ( τ ) = \frac{1}{{N}_{i}} {\sum }_{i = 1}^{{N}_{i}} {[ y ( {t}_{i} + τ ) - y ( {t}_{i} ) ]}^{2}$$

$$MSD ( τ ) = {MSD}_{x} ( τ ) + {MSD}_{y} ( τ )$$

Where Nᵢ is the number of observations corresponding to time lag τ. For a given recorded particle trajectory, more observations may be extracted for a smaller time-lag than a larger time-lag. Accordingly, the uncertainty in MSD increases with increasing time-lag. For the entire set of particles observed in the experiment in FIG. 7, the ensemble average MSD for a given time lag τ is calculated as the average of the individual particle MSD for that time lag. The results shown in FIG. 7 demonstrate that particle MSD may be used to differentiate between different concentrations of analyte. MSD of particles decreases with increasing analyte concentration both when particles are observed immediately after they settle and when residual particles on the surface are observed after weakly bound particles are removed via slide inversion and gravity.

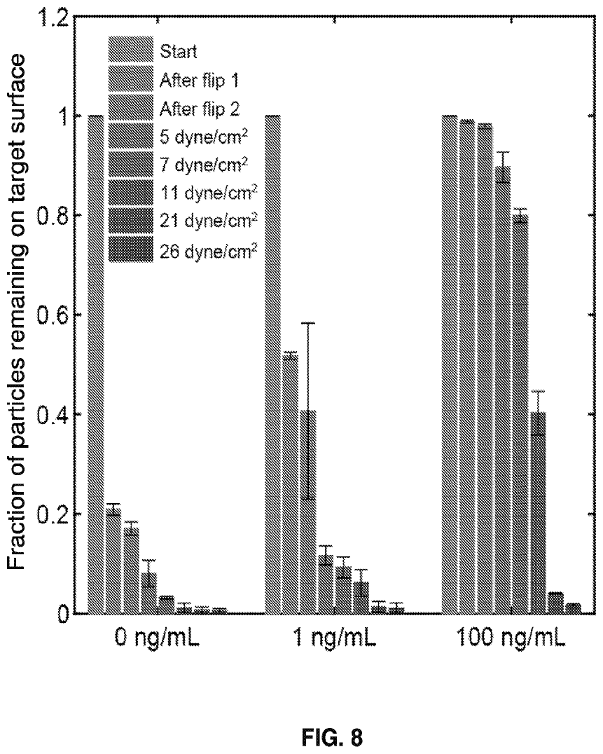

FIG. 8 shows data from gravity and vibration-induced dissociation assays. In these assays, particles are incubated with an analyte solution in the container and allowed to settle under gravity onto an affinity agent-functionalized target surface. The surface is then inverted to allow weakly- or untethered particles to fall off under the influence of gravity. Using a coin vibration motor (CVM) externally adhered to the surface, the surface is vibrated at different intensities by applying a voltage across the motor, which induces a shear force on the particles that remain tethered to the surface. This shear is modulated by the CVM vibration intensity and can be tuned such that particles that are nonspecifically bound may be removed, while the strongly tethered, analyte-bound particles may remain on the surface. Particle quantification and subsequent analyte concentration may be determined by counting the number of particles that remain on the surface.

The particles in FIG. 8 are 4.5 μm superparamagnetic particles coated with an affinity agent and incubated with either 0, 1 or 100 ng/ml of analyte, washed, and allowed to settle onto an affinity agent-functionalized, antifouling polymer-coated glass slide. Brightfield microscopy was used to count the particles on the target surface after settling (“start” condition), after inverting the slide once and twice (“after flip 1” and “after flip 2” condition), and after vibration of the slide with a CVM that produces a specific shear stress (conditions specified by a certain dyne/cm²). In some embodiments, quantification of the number of particles on the surface after each condition and comparison with the starting number of particles may distinguish between different concentrations of analyte in the original solution. For example, there is an observably different concentration of particles on the surface between 0, 1 and 100 ng/mL in the “after flip” condition. The fraction of particles that remain on the surface may increase with increasing analyte concentration because of the presence of specific interactions between analyte-bound particles and the affinity agent-functionalized surface.

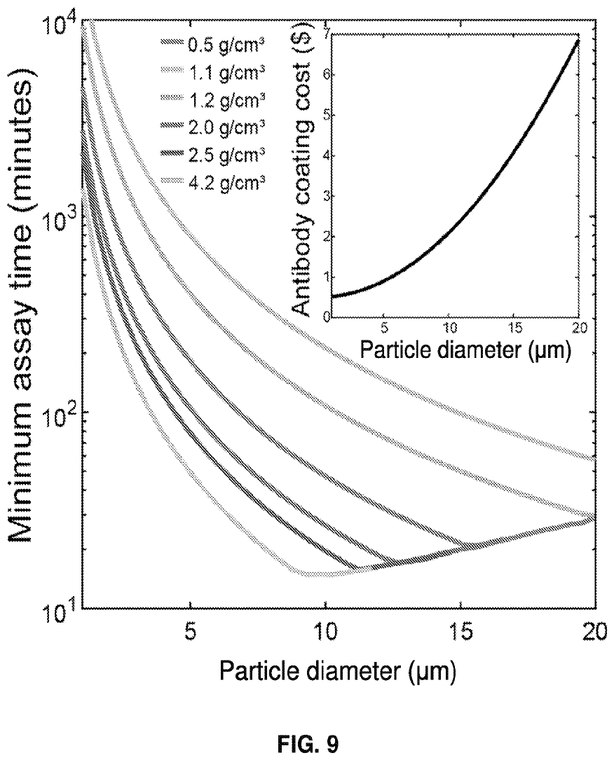

FIG. 9 shows an exemplary graph of simulated assay time as a function of various assay properties, such as particle diameter and density, for an exemplary testing system for detecting SARS-COV-2 virions in a fluid using silica particles coated with IgG antibodies. The simulations represented in FIG. 9 have been calculated for a total sample fluid volume of 1 mL, 1 million particles, virion concentration of 10⁴ per mL (representative of the minimum viral load measured in hospitalized patients), a 2.7 cm settling distance, 80.2° target surface slope having a length of 1 cm, limited by sedimentation and rolling times of less than 30 minutes and an antibody cost of under $3.

As shown in FIG. 9, each parameter has a different effect. For example, with gravity as the external driving force, particle density and diameter, combined with fluid viscosity, may govern the settling speed of the particles, which, combined with height of the fluid column above the target surface inside the container, may determine the settling time of the particles. The particle velocity, shape, size, and settling time, in conjunction with binding and analyte properties (such as size and diffusivity), may govern concentration of analyte captured by particles. Analyte capture increases with increasing number of particles (for the same fluid volume). The particle size, density, stiffness, slope of the surface, nature of the surfaces, etc. govern how fast particles roll on the surface. The length of the surface in the direction of particle motion may govern the contact time and the probability of binding between an analyte-bound particle and the surface. Particle size, density, and the nature of the binding may govern whether the particle motion will be affected by binding to the surface, or whether it may be bound on the surface.

It should be appreciated that although larger particles and denser materials yield faster assays, they may also increase antibody costs due to greater total surface area for the same number of particles (see FIG. 9 inset). Considering this tradeoff between sensitivity, cost, and time optimizes the SARS-COV-2 virion detection assay by using 10 μm silica particles. Combined with an estimated silica particle cost of $2.50 and considering considerably lower costs of economies of scale, the testing system may very likely cost less than $10 per test. It should be appreciated that although a reader having a microscope may cost $20-$50, based on current prices of low-power microscopes that involve a similar degree of optical and mechanical complexity, the use of smartphones as the reader are also contemplated.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks. In some embodiments, cloud computing infrastructure may be employed for processing purposes.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. It is also contemplated that machine learning based approaches may be used to analyze data from the sensors (such as a sequence of images) and in conjunction with other inputs such as type of sample, type of test, magnitude of secondary force, or patient data, may be used to infer a result such as concentration of analyte or estimation of the probability of a certain disease condition, diagnosis, or prognosis of the patient.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A method of analyte detection, the method comprising:
providing a volume having a first plurality of particles;
introducing a first analyte solution to the volume, wherein the first plurality of particles are configured to react with a first analyte when present in the first analyte solution;
applying an external force to the volume to urge the first plurality of particles to move through the first analyte solution; and
allowing the first plurality of particles to settle on a first portion of a target surface, wherein the first portion of the target surface is configured to bind the first plurality of particles when the first analyte is present in the first analyte solution.

2. The method of claim 1, further comprising determining the presence of the first analyte.

3. The method of claim 1, further comprising determining the concentration of the first analyte.

4. The method of claim 1, wherein the external force is one selected from the group of gravitational, electrokinetic, electrohydrodynamic, dielectrophoretic, acoustic, magnetohydrodynamic, thermal convection, optical, radiation, and magnetic.

5. The method of claim 1, further comprising applying the external force to the volume to urge the first plurality of particles to move through the first analyte solution at a rate faster than a diffusion limited rate.

6. The method of claim 1, further comprising:
applying the external force to the volume to urge a second plurality of particles to move through the first analyte solution; and
allowing the second plurality of particles to settle on a second portion of the target surface, wherein the second portion of the target surface is configured to bind with the second plurality of particles when a second analyte is present in the first analyte solution,

7. The method of claim 1, further comprising applying a secondary external force to the volume to dislodge a portion of the first plurality of particles nonspecifically bound to the target surface when present.

8. A method of analyte detection, the method comprising:
obtaining a sequence of images of a surface in a volume, the surface having a first plurality of particles disposed on the surface, wherein the first plurality of particles are configured to react with a first analyte when present, and wherein the surface is configured to bind to the first plurality of particles when the first analyte is present;
measuring relative displacement of the first plurality of particles based at least in part on the sequence of images; and
determining the presence of the first analyte based at least in part on the relative displacement of the first plurality of particles.

9. The method of claim 8, further comprising determining the concentration of the first analyte based at least in part on the relative displacement of the first plurality of particles.

10. The method of claim 8, further comprising determining the fraction of the first plurality of particles nonspecifically bound to the surface based at least in part on the relative displacement of the first plurality of particles.

11. The method of claim 8, wherein the surface includes a second plurality of particles disposed on the surface, wherein the second plurality of particles are configured to react with a second analyte when present, and wherein the method further comprises:
measuring relative displacement of the second plurality of particles based at least in part on the sequence of images; and
determining the presence of the second analyte based at least in part on the relative displacement of the second plurality of particles.

12. A system for analyte detection, the system comprising:
a volume configured to contain a first analyte solution;
a first plurality of particles arranged in the volume, the first plurality of particles configured to react with a first analyte when present in the first analyte solution; and
a first target surface configured to bind with the first plurality of particles when the first analyte is present in the first analyte solution,
wherein the first plurality of particles is configured to move through the first analyte solution toward the first target surface.

13. The system of claim 12, wherein the first plurality of particles are configured to move through the first analyte solution when exposed to an external force, and wherein the external force is one selected from the group of gravitational, electrokinetic, electrohydrodynamic, dielectrophoretic, acoustic, magnetohydrodynamic, thermal convection, optical, radiation, and magnetic.

14. The system of claim 13, wherein the external force is configured to urge the first plurality of particles through the first analyte solution at a rate greater faster than a diffusion limited rate.

15. The system of claim 12, wherein the first plurality of particles are formed at least partially from one or more materials from the group of metallic, polymeric, ceramic, magnetic, and paramagnetic.

16. The system of claim 12, wherein the first plurality of particles are at least partially coated with a first binding ligand and/or coating configured to attract the first analyte.

17. The system of claim 12, further comprising an antifouling coating disposed on the first target surface.

18. The system of claim 12, wherein the first target surface is at least partially coated with a first binding ligand and/or coating configured to attract the first analyte.

19. The system of claim 12, further comprising:
a reader configured to collect a sequence of images of the first target surface; and
an image analysis system configured to analyze the sequence of images.

20. The system of claim 12, further comprising:
a second plurality of particles arranged in the volume, the second plurality of particles configured to react with a second analyte when present in the first analyte solution, and
a second target surface, wherein the second portion of the target surface is configured to bind with the second plurality of particles when the second analyte is present in the first analyte solution.

21. The system of claim 12, wherein the first plurality of particles is configured to bind to the first target surface when the first analyte is present in the first analyte solution.

22. The system of claim 12, wherein the presence of the first analyte in the first analyte solution induces a binding reaction between the first target surface and the first plurality of particles.
